(19) United States
(12) Patent Application Publication
(10) Pub. No.: US 2017/0037419 A1
(43) Pub. Date: Feb. 9, 2017

(54) Methods And Materials For Producing Enhanced Sugar, Starch, Oil, And Cellulose Output Traits In Crop Plants

(71) Applicant: The Arizona Board of Regents On Behalf Of The University Of Arizona, Tucson, AZ (US)

(72) Inventor: Eliot M. Herman, Tucson, AZ (US)

(73) Assignee: The Arizona Board of Regents On Behalf Of The University Of Arizona, Tucson, AZ (US)

(21) Appl. No.: 15/124,705

(22) PCT Filed: Mar. 12, 2015
(86) PCT No.: PCT/US15/20254

§ 371 Date: Sep. 9, 2016

Related U.S. Application Data
(60) Provisional application No. 61/951,996, filed on Mar. 12, 2014.

(51) Int. Cl. C12N 15/82; C12N 9/82

(52) U.S. Cl. CPC C12N 15/8245; C12N 15/8247; C12N 15/8246; C12N 9/82; C12Y 305/01001

(57) ABSTRACT

Described are crop-related materials and methods for metabolic engineering. Certain aspects of the invention include applications in food production, carbon sequestration, and biofuel production. Described are methods of enhancing plant traits for increased production of sugar, starch, cellulose, and oil. Described methods include altering cytosolic asparagine to promote production of non-nitrogenous plant compounds.

### CROSS-REFERENCE TO RELATED APPLICATIONS

This application claims the benefit of U.S. Provisional Application No. 61/951,996 filed Mar. 12, 2014, the entire disclosure of which is expressly incorporated herein by reference for all purposes.

### REFERENCE TO SEQUENCE LISTING SUBMITTED VIA EFS-WEB

This application is being filed electronically via the USPTO EFS-WEB server, as authorized and set forth in MPEP§1730 II.B.2(a)(A), and this electronic filing includes an electronically submitted sequence (SEQ ID) listing. The entire content of this sequence listing is herein incorporated by reference for all purposes. The sequence listing is identified on the electronically filed .txt file as follows: 53-55555-UA14-047_SL.txt, created on Mar. 10, 2015 and is 4,712 bytes in size.

### BACKGROUND OF THE INVENTION

There is a metabolic interplay of carbon and nitrogen that sets the stage for a plant's productivity and carbon allocation among all its constituents. While carbon input is freely available as CO₂, nitrogen input is different. Elemental nitrogen is freely available, but 98% of the fixed soil nitrogen is generally unavailable to plants, being bound into already accumulated biomass.

Plants depend on a variety of microbial systems including symbiotic bacteria to reduce nitrogen into forms available for uptake as nitrates or ammonium, especially as applied fertilizer. In a biogeographic and ecological context, plants are highly competitive so, for example, when nitrogen is limiting, plants may employ mechanisms such as enhanced root growth to enlarge the uptake area in order to compete for resources. Another mechanism employed when nitrogen is limiting is for plants to adapt to minimize nitrogen needs by shifting toward a composition with a higher C:N and to recycle assimilated nitrogen through more stringent organ/cellular triage.

Although the paradigm of nitrogen-limited growth—derived from decades of nitrogen stress research—is one of retarded growth and development, the real world situation is more complex. In practice, some plants adapt and thrive under nitrogen limitation, while others are significantly impaired. This is advantageous for competitive selection. With plant-plant competition for nitrogen resources in nitrogen-limited environments, there is selection pressure for a plant's physiology to be more broadly tolerant of varying nitrogen inputs and for the plants to favor a higher carbon to nitrogen composition ratio, accumulating greater carbonaceous reserves than might occur for growth under conditions of nitrogen surplus. In this context, a plant gains a selective advantage with the ability to exhibit a broader C:N composition as an input trait that would allow it to adapt to different environments and to expand the plant's range. The plant's compositional plasticity enables it to complete its life cycle under varying environments and to thereby competitively expand its range.

A key compositional control occurs with the shuttling of accumulated nitrogen between plant organs in response to limited nitrogen availability. Plants translocate accumulated nitrogen reserves from older to newer tissues of the plant by programmed turnover of cellular constituents. During the senescence of older tissues/organs, autophagy results in shuttling of derived nutrients to younger tissues, and is a primary means for translocating accumulated nitrogen within the plant. Under conditions of limited nitrogen, this mechanism becomes less selective and systemic, with the plant's cells triaging its constituents and degrading various components to provide nutrients required for essential plant growth and prioritizing completion of the life cycle. Such plants usually manifest stunted growth, limited reproductive output, and present with tissues that possess higher C:N ratios. Rebalancing composition generally involves reducing nitrogenous molecule accumulation, especially protein, and favoring accumulation of carbon-rich glycans, resulting in plant development that is focused on the singular goal of completing a reproductive cycle to disperse its progeny to better conditions.

The fixed carbon flux of plants is allocated to plant growth and development, and to accumulate stored substances as nutrient reserves for life-cycle processes. Many of these stored reserves in crop plants constitute the key agricultural commodities of biomass, starch, sugar, and oil. Improving the efficiency of the carbon flux is important for more effective and efficient land, water, and fertilizer use to meet the needs of a growing population. One possible strategy is nitrogen limitation. Nitrogen limitation results in systems rebalancing, leading to a decrease in protein content through limited synthesis and induced degradation, and results in the accumulation of non-nitrogenous compounds, such as, sugars, starch, cellulose, and oil. However, nitrogen limitation is not a feasible means to encourage higher C:N ratios in plant tissues, as such strategies severely impair plant growth and reproductive output. A more sophisticated approach is required to reprogram the plant's allocation of fixed carbon to produce plants that perform normally in an agronomic context but possess economically valuable enhanced carbonaceous solid compositions.

### SUMMARY OF THE INVENTION

To improve the efficiency and yield of carbonaceous products, a biotechnology engineering strategy has been developed that has demonstrated by reducing nitrogen source, by destroying free cytoplasmic asparagine, a primary ammonium source in plants, induces a plant to systemically and/or organ-specifically to produce an output trait that rebalances carbon composition to increase sugars and sugar-derived polymers.

In a particular embodiment described herein is a method for redirecting a plant's allocation of fixed carbon toward carbohydrate polymers relative to a control plant, comprising increasing expression in a plant a nucleic acid encoding a polypeptide having at least 70% sequence identity to the amino acid sequence of SEQ ID NO: 2. In other embodiments, the nucleic acid encoding the polypeptide has a sequence identity to the amino acid sequence of SEQ ID NO: 2 selected from the group consisting of: at least 70%; at least 75%; at least 80%; at least 85%; at least 90%; at least 95%; at least 96%; at least 97%; at least 98%; at least 99%; and 100%. In yet another embodiment, the nucleic acid comprises the nucleotide sequence of SEQ ID NO: 1 or encode a polypeptide comprising the amino acid sequence of SEQ ID NO: 2.

In certain embodiments, the he expression of the nucleic acid is increases by introducing and expressing the nucleic acid in the plant.

In another embodiment, the plant comprises an enhanced level of fixed carbohydrate relative to a control plant in at least one form of carbohydrate polymer selected from the group consisting of: sugars; starch; cellulose; and oil. Ideally, the redirection of the plant's allocation of fixed carbon towards carbohydrate polymers relative to a control plant occurs under non-stress conditions.

In another embodiment described herein, the nucleic acid is operably linked to a promoter. The promoter may be a ubiquitous constitutive promoter, such as the cauliflower mosaic virus 35s promoter, or a tissue specific promoter, specific for a tissue selected from the group of tissues consisting of: root; taproot; tuber; stem; leaf; petal; fruit; and seed. In yet other embodiments, the tissue specific promoter is selected from the group consisting of: patatin tuber-specific promoter; E8 tomato fruit-specific promoter; SRD1 taproot-specific promoter; Mll taproot-specific promoter; His1-r taproot specific promoter; Tlp taproot specific promoter; oleosin seed-specific promoter; and glycinin seed-specific promoter.

In another embodiment described herein, a second copy of the nucleic acid is introduced and expressed in the plant, wherein the second copy of the nucleic acid is operably linked to a second promoter. In one embodiment, one copy of the nucleic acid is operably linked to a tissue non-specific promoter and the other copy of the nucleic acid is operably linked to a tissue-specific promoter.

The methods described herein may be used to accelerate a plants growth rate and/or life cycle.

In certain embodiments, the plant is selected from the group consisting of: soybean; potato; tomato; tobacco; Camelina spp; maize; carrot; switchgrass; sugar beet; cassava; sweet potato; yam; Brachypodium; onion; safflower; sunflower; canola (rapeseed); hemp; cotton; sesame; peanut; flax; rice; wheat; and oats.

In yet other embodiments described herein are plants obtained by methods described herein. In certain embodiments, the plants or parts thereof comprise 20-50% greater cellulose content per unit mass greater than a wild-type control plant grown in parallel; 55-75% less free asparagine than a wild-type control plant grown in parallel; a 2-3 fold increase in sugar content relative to a wild-type control plant grown in parallel; an at least 1% increase in oil content relative to a wild-type control plant grown in parallel; and/or a lifecycle accelerated by 5-20%.

In a particular embodiment described herein is a construct comprising a first copy of a nucleic acid encoding a polypeptide having at least 70% sequence identity to the amino acid sequence of SEQ ID NO: 2, one or more control sequences capable of driving expression of the first copy of the nucleic acid, and optionally a transcription termination sequence.

In another embodiment described herein, the construct further comprising a second copy of the nucleic acid sequence and one or more control sequences capable of driving expression of the second copy of the nucleic acid, wherein the one or more control sequences capable of driving expression of the second copy of the nucleic acid are different from the one or more control sequences capable of driving expression of the first copy of the nucleic acid sequence.

Also described herein are plants, plant parts, and plant cells comprising a construct described herein, and harvestable parts of plants comprising the construct, wherein the harvestable parts are selected from the group consisting of: shoot biomass; fruits; roots; taproot; and seeds, and wherein the harvestable parts comprise the construct.

In a particular embodiment described herein is a method for making a plant having altered fixed carbon allocation relative to a control plant, comprising transforming a plant, plant part, or plant cell with a construct described herein.

In a particular embodiment described herein is a method for the production of a transgenic plant having increased sugar, starch, cellulose, and/or oil content relative to a control plant, comprising introducing and expressing in a plant or plant cell a nucleic acid encoding a polypeptide having at least 70% sequence identity to the amino acid sequence of SEQ ID NO: 2, and cultivating the plant or plant cell under conditions promoting plant growth and development.

Also described herein are plants, plant parts, and plant cells produced by a transgenic method described herein, harvestable parts of the produced transgenic plants, and products obtained from the transgenic plants.

In a particular embodiment described herein is a method for increasing oil production in algae, comprising increasing expression in algae a nucleic acid encoding a poly nucleotide having at least 70% sequence identity to the amino acid sequence of SEQ ID NO: 2.

In another embodiment described herein, the algae's growth rate and/or life cycle is accelerated relative to a control.

In yet other embodiments described herein are products obtained from the algae described herein. In certain embodiments, the products are oils.

In a particular embodiment described herein is a method for scavenging environmental CO₂, comprising increasing expression in algae a nucleic acid encoding a poly nucleotide having at least 70% sequence identity to the amino acid sequence of SEQ ID NO: 2, and scavenging environmental CO₂.

Various objects and advantages of this invention will become apparent to those skilled in the art from the following detailed description, when read in light of the accompanying figures.

### BRIEF DESCRIPTION OF THE FIGURES

The patent or application file may contain one or more drawings executed in color and/or one or more photographs. Copies of this patent or patent application publication with color drawing(s) and/or photograph(s) will be provided by the Patent Office upon request and payment of the necessary fee.

FIGS. 1A-1C: Soybean Asnase is a cytosolic enzyme. FIG. 1A) Diagram of Asnase expression constructs transferred to onion and tobacco cells. FIG. 1B) Photographs of onion cells bombarded by either a 35S-Asnase(potato)-YFP construct (left) or a 35S-Asnase(soybean)-YFP construct (right), both showing a cytosolic distribution of the ectopic Asnase enzyme. FIG. 1C) Photographs of tobacco cells bombarded by either a 35S-Asnase(potato)-YFP construct (left) or a 35S-Asnase(soybean)-YFP construct (right), both showing a cytosolic distribution of the ectopic Asnase enzyme.

FIGS. 2A-2F: FIG. 2A) Photograph showing the accelerated growth changes that result from the ectopic expression of 35S-Asnase as stable homozygotes in tobacco as a side-by-side comparison with wild-type plants grown in parallel as a control. FIG. 2B) Photographs showing transgenic tobacco plants (plants on left in each photo) and wild-type plants (plants on right in each photo) grown in parallel in either low light of high light conditions. FIG. 2C-2F) Bar graphs showing differences in leaf dry mass (FIG. 2C), specific leaf weight (FIG. 2D), leaf area (FIG. 2E), and number of leaves per plant (FIG. 2F) between transgenic and wild-type tobacco plants grown in either low light (LL) or high light (HL) conditions. *=p<0.05; **=p<0.01.

FIGS. 3A-3E: Bar graphs showing the 75% net reduction of free Asn (FIG. 3A) in tobacco plants expressing ectopic Asnase, which in turn induces a reduction in protein (FIG. 3B) and chlorophyll (FIG. 3C) content, while in parallel increasing starch content (FIG. 3D) about two fold. FIG. 3E) Bar graph showing plant heights of tobacco plants expressing ectopic Asnase compared to wild-type controls grown in parallel The results shown represent homozygous plants from two separate events.

FIGS. 4A-4B: FIG. 4A) Table showing a selection of metabolomic results from tobacco plants expressing ectopic Asnase, outlining the net decrease in amino acid abundance and the net increase in the abundance of some free sugars. FIG. 4B) Bar graph showing the distribution and abundance of total amino acids in tobacco plants expressing ectopic Asnase and wild-type plants.

FIG. 5: Principle component analysis map showing the differences in metabolomics profiles of wild type and ectopic Asnase-expressing tobacco leaves. The map outlines the configuration of the metabolomics profile of the Asnase expressing plants, and shows that the leaves of the Asnase-expressing plants are metabolically distinct from controls grown in parallel, emphasizing that reducing ammonium input into metabolism results in a large scale remodeling of the leaf steady state metabolism.

FIG. 6: Photograph showing the growth differences of hydroponic wild-type and ectopic Asnase-expressing tobacco plants.

FIG. 7: Photograph showing the morphology and mass differences of hydroponic wild-type and Asnase expressing tobacco plants.

FIGS. 8A-8B: FIG. 8A) Bar graph showing quantification of the root mass differences in the hydroponic wild-type and Asnase expressing tobacco plants. FIG. 8B) Bar graph showing differences in the weight of roots as a percentage of total plant dry mass of wild-type and Asnase expressing tobacco plants grown in either low light (LL) or high light (HL) conditions. **=p<0.01.

FIG. 9: Bar graph showing ¹¹C incorporation results. Carbon fixation per unit mass in ectopic Asnase-expressing tobacco plants is about 33% higher than that of the wild type.

FIG. 10: Bar graph showing that carbon fixation in ectopic Asnase-expressing plants results in 40% greater cellulose content per unit mass in the Asnase-expressing plants compared to wild type. The increase in carbon fixation results in about a 15% increase of carbon flux into cellulose in the Asnase plants compared to wild-type.

FIG. 11: Photographs showing that potatoes expressing ectopic soybean or potato Asnase appear to be overtly identical to wild-type.

FIG. 12: Bar graph showing the decrease in asparagine content of potato tubers expressing potato Asnase (line 14.9, 14.50) and soybean Asnase (15.4 and 15.52) compared to control cv. Atlantic conventional wild-type. Note that all transgenic lines expressing either Asnase transgene exhibit 50% less free Asnase than the control (WT).

FIG. 13: Two-dimensional gels of total potato tuber proteins from potato and soybean Asnase transgenics compared with conventional cv. Atlantic. The in situ hydrolysis of free asparagine does not significantly alter the proteome of the potato tuber.

FIG. 14: A heat map of the relative abundance of the non-targeted metabolome set from lysates of conventional cv. Atlantic and ectopic potato and soybean Asnase-expressing tubers. Although all of the transgenic tuber lines exhibited >50% reduction in free asparagine, the overall abundance of non-targeted metabolites appeared to fall into at least two distinct patterns that were different from the conventional Atlantic cultivar. These two patterns are represented in this heat map by the similarity of potato Asnase line 14.9 and soybean line 15.40 being more similar to the other, while potato Asnase line 14.40 and soybean Asnase line 15.52 were also more similar to each other.

FIG. 15: Diagram showing the metabolomic effect of the ectopic expression of soybean and potato Asnase on the immediate down-stream amino acid pathway in potato tuber. Asparagine hydrolysis results in a uniform, though slight, reduction in aspartate content in all lines; however, further down-stream alterations in amino acid content is variable, showing the complexities of amino acid synthesis regulation and feedback controls. The individual bar graphs show the relative changes in amino acid content of the four transgenic lines compared to the conventional wild-type correlated with the relevant amino acid biosynthesis pathway. The large red arrow shows the pathway position where the over-expression of potato and soybean Asnase destroys free asparagine and releases NH₄ ions (shown in red) that under normal circumstances would transfer N to form glutamine.

FIG. 16: Diagram showing the metabolomic consequences of destroying free asparagine on further down-stream arginine and polyamine biosynthesis. The decrease in free asparagine shown decreases the availability of NH₄ for N transfer to other compounds. This is reflected in the general decrease of arginine and arginine-related species and as increases in free glutamate in some of the potato tuber samples.

FIG. 17: A summary diagram of the averaged metabolomics results showing the changes in intermediate metabolism encompassing the major amino acids and related substances, citric acid cycle, and sugar/glycan metabolism. Decreasing NH₄ availability by Asn hydrolysis results in an over-all decrease in abundance of free amino acids and related substances with the exception of glutamate. This results in a general increase in abundance of substances that do not contain intrinsic nitrogen, including citric acid cycle components, fatty acids, glucose, as well as maltodextrin polymers. Note the general reduction of amino acids and the relative increase in sugars, showing tubers exhibit similar results compared with the tobacco leaves.

FIG. 18: A photograph of a tomato plant without (left) and with (right) and Asnase overexpression.

FIG. 19: A photograph of a GUS control tomato plant (left) and a tomato plant with (right) Asnase overexpression.

FIG. 20: Fluorescence analysis curve showing segregation of homozygous and heterozygous soybean plants expressing L-asparaginase (Asnase) under the control of the glycinin promoter. The RT-PCR assay relied on gene dosage to differentiate homozygous and heterozygous plants.

FIG. 21: Principle component analysis map showing the differences in metabolomics profiles of wild-type (standard tobacco) and ectopic Asnase-expressing tobacco seeds, under control of the ubiquitous 35S promoter, targeted to vacuoles, or under control of a seed-specific promoter. The map outlines the configuration of the metabolomics profile of the Asnase expressing plants, and shows that the seeds of the tobacco plants wherein Asnase-expression is driven by a seed specific promoter are metabolically distinct from controls and tobacco plants wherein Asnase-expression is vacuole-specific, or driven by the ubiquitous 35S grown in parallel.

### DETAILED DESCRIPTION

Plant organs contain a variety of biochemical constituents specified by genetic program that can be modified to varying degrees by extrinsic environmental and nutritional cues. Among these, nitrogen limitation results in systems rebalancing leading to a decrease in protein content through limited synthesis and induced degradation, resulting in the accumulation of non-nitrogenous compounds, such as, sugars, starch, cellulose, and oil, as a redirected means of sequestering excess carbon flux. As a consequence, it is N rather that C that is the currency of life, with N availability specifying the compositional output trait by manipulating carbon-rich substance accumulation. These non-nitrogenous carbonaceous compounds that are accumulated in response to nitrogen limitation are desirable as they are the biochemical feedstocks for bioenergy and foodstuff production. However, as a strategy, nitrogen starvation to remodel plant composition has an obvious flaw in that, while the tissue may contain more carbohydrate per unit mass of nitrogen, stressed plants grow poorly so that the net loss in biomass will outweigh the benefits of improved fermentation quality or other compositional traits.

Asparagine (Asn) is one of the primary nitrogen transport molecules and provides ammonium input into a plant's metabolome. As shown and described herein, manipulating free cellular Asn content alters the plant's perception of nitrogen status to provoke changes in the systems biology of the plant. Limiting free Asn induces the plant to reprogram its metabolism to remodel its growth and composition into increasing the ratio of carbon to nitrogen.

New plant genotypes are described herein that mimic the genotypes of plants grown in nitrogen-limited environments that manifest the phenotype of higher C:N ratios. Altering perceived source input, yields a range of high C:N ratio phenotypes that, for instance, can be leveraged to create, for example, soybean having higher oil content, tomatoes having higher fruit glycan content, or a biofuel-content-optimized biomass line of crops. By genetically manipulating the plant's perception of nitrogen status, and therefore apparent nitrogen availability, the results show it is feasible to induce plants to redirect a fraction of its carbon flux from protein to carbohydrate polymers.

As mentioned above, Asn occupies a key position in plant nitrogen metabolism. Low carbohydrate content stimulates Asn synthesis that is the inverse of the other amino acids. Asn has a feedback inhibition of nitrate reduction when systemic sugar content is depleted. The role of Asn content in regulating metabolism indicates that plants sense Asn content as a proxy for available ammonium and, if the Asn content is reduced, plants rebalance the systemic carbon flux to favor increased carbohydrate and polymers paralleling the compositional changes of nitrogen-limited plants. By reducing Asn content, the resulting cascade of metabolic, transcriptomic, and overt changes alter the plant in ways favorable to desired output traits for biofuel biomass and carbonaceous food products.

Methods to alter Asn content encompass inhibiting Asn synthesis or destroying cellular free Asn by in situ hydrolysis. Hydrolysis of free Asn does not impair any major metabolic pathway and the Asnase reduction is not complete. Rather, the content of free Asn is a sum of complex factors, including competition for Asn substrate and regulation of compensatory synthesis. As shown in the results described herein, induced hydrolysis of Asn significantly reduces the concentration of free Asn, resulting in useful plant phenotypes.

Plants widely express the gene for L-Asparaginase (hereafter termed Asnase). Asnase has two subunits derived from a common precursor protein by autocatalysis. Plant Asnase has a solved crystal structure. There two major forms of Asnase, K⁺-dependent and K⁺-independent. The sequences from diverse plants cluster into these two major groups. Asnase is cytosolic, as confirmed by YFP fusions described herein, and differentially expressed in plant tissues and organs.

Asnase is highly expressed in those plant tissues that have a large flux of nitrogen, such as seeds mobilizing storage proteins during post-germination growth. Of those Asnases with reported enzyme kinetics, Asnases derived from legume seeds are among those with the highest specific activity. An Asnase gene expressed in soybean seeds was thus selected as an experimental model for using Asnase activity to modulate the perceived nitrogen status in target plants.

Reducing the in situ concentration of free Asn by hydrolysis results in the plant accelerating its growth rate and shifting its allocation of fixed carbon to favor glycans. This results in the excess accumulation of glycan polymers, in particular, starch, cellulose, and oil, as well as an increase in free sugars. The resulting phenotype is an example of a complex class of traits. Engineering plants to maintain, or even enhance, agronomic output traits while using less input thereby increases sustainability, i.e., ‘making more from less.’ This technology can be used to reprogram plants and/or specific plant organs to accumulate enhanced sugar content, glycan polymers derived from sugars, and/or oils.

Production of Plants with Higher C:N Ratios by Transgenic Methods.

According to one aspect of the present invention, a nucleic acid (preferably DNA) sequence encoding an Asnase gene may be used for the production of a plant having an altered allocation of fixed carbon, wherein the ratio of C:N in the plant or a particular organ within a plant is higher than in a control plant. In this aspect, the invention provides for introduction of a nucleic acid sequence encoding an Asnase into a plant that would benefit from reprogramming of its fixed carbon allocation. One suitable plant capable of providing a nucleic acid sequence encoding an Asnase having high specificity is soybean (Glycine max; SEQ ID NO: 1). Nucleic acid sequences encoding Asnase derived from other plants may also be used. Asnases of legumes (including soybean) are known to have high specificity, and are therefore suitable sources of Asnase-encoding nucleic acid sequences. Nucleic acid sequences derived from soybean and other legumes may be introduced into many plants to redirect fixed carbon flux to carbohydrate polymer, including but not limited to soybean (Glycine max), carrot (Daucus carota), sugar beet (Beta vulgaris), cassava (Manihot esculenta), potato (Solanum tuberosum), yam (Dioscorea spp.), sweet potato (Ipomoea batatas), maize (Zea mays), switchgrass (Panicum virgatum), cotton (Gossypium spp.), sunflower (Helianthus annuus), canola (rapeseed; Brassica napus), sesame (Sesamum indicum), flax (Linum usitatissimum), safflower (Carthamus tinctorius), peanut (Arachis hypogaea), and Camelina spp. Trees such as pine (Pinus spp.), may also benefit from the materials and methods disclosed and described herein. In certain embodiments, an Asnase-encoding nucleic acid sequence from a particular type of plant may be transformed into an individual of the same type, thereby resulting in overexpression of Asnase in the plant (see, for example, potatoes of Example 3).

A suitable nucleic acid sequence encoding Asnase (e.g., SEQ ID NO: 1) may be transferred to a suitable recipient plant by any method available. For instance, the nucleic acid sequence may be transferred by transformation or by any other nucleic acid transfer system, optionally followed by selection of offspring plants comprising the nucleic acid sequence and exhibiting a desired altered fixed carbon accumulation, as evidenced by, for example, increased starch, sugar, and/or oil accumulation in the overall plant or a target plant organ, or overall biomass.

For transgenic methods of transferring a nucleic acid sequence encoding an Asnase, the sequence may be isolated from a donor plant by methods known in the art, or the sequence may be generated and/or amplified by methods known in the art, for example, by polymerase chain reaction (PCR), molecular cloning, and solid-phase DNA synthesis. In certain embodiments, cDNA encoding the desired Asnase is generated by methods well known in the art. The thus isolated, generated, and/or amplified nucleic acid sequence may be transferred to the recipient plant by transgenic methods, for instance by means of a vector or construct, in a gamete, or in any other suitable transfer element, such as a ballistic particle coated with said nucleic acid sequence.

Plant transformation generally involves the construction of a recombinant expression vector or construct that will function in plant cells. In the present invention, such a construct comprises a nucleic acid sequence that encodes an Asnase polypeptide, wherein the nucleic acid sequence is under control of, or operatively linked to, a regulatory element such as a promoter. The construct may contain one or more such operably linked gene/regulatory element combinations, provided that at least one of the genes contained in the combinations encodes for Asnase. The construct may be in the form of a plasmid, and can be used alone or in combination with other plasmids to provide transgenic plants that have a desired altered fixed carbon allocation, using transformation methods known in the art, such as the Agrobacterium transformation system.

“Recombinant” as it refers to an expression vector or construct, means a DNA molecule that is made by combination of two otherwise separated segments of DNA, e.g., by chemical synthesis or by the manipulation of isolated segments of nucleic acids by genetic engineering. Recombinant DNA can include exogenous DNA or simply a manipulated native DNA. Recombinant DNA for expressing a protein in a plant is typically provided as an expression cassette which has a promoter that is active in plant cells operably linked to DNA encoding a protein of interest.

“Regulatory element”, “control sequence” and “promoter” are all used interchangeably herein and are to be taken in a broad context to refer to regulatory nucleic acid sequences capable of effecting expression of the sequences to which they are ligated. The term “promoter” typically refers to a nucleic acid control sequence located upstream from the transcriptional start of a gene and which is involved in recognizing and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. The term “regulatory element” also encompasses a synthetic fusion molecule or derivative that confers, activates or enhances expression of a nucleic acid molecule in a cell, tissue or organ.

As used herein, a “promoter” comprises regulatory elements, which mediate the expression of a coding sequence segment in plant cells. The promoter need not be of plant origin, but may originate from viruses or micro-organisms, for example from viruses which attack plant cells. The promoter can also originate from a plant cell, e.g. from the plant which is transformed with the nucleic acid sequence to be expressed in the methods described herein.

Promoters useful for driving expression a nucleic acid are preferably operably linked to the nucleic acid. As used herein, “operably linked” refers to a functional linkage between the promoter sequence and the nucleic acid of interest, such that the promoter sequence is able to initiate transcription of the nucleic acid of interest.

Promoters may be constitutive promoters or inducible promoters. Constitutive promoters are transcriptionally active during most phases of plant growth and developments, and under most environmental conditions, in at least one cell, tissue, or organ. Constitutive promoters for driving nucleic acid expression in transformed plants include, but are not limited to CaMV 35S promoter, CaMV 19S promoter, Ubiquitin promoter, Maize H3 histone promoter, Alfalfa H3 histone promoter, RUBISCO small subunit promoter, and Super promoter. Those of ordinary skill in the art that other constitutive promoters effective in plants may be used.

Constitutive promoters may be ubiquitous promoters, which are active in substantially all tissues or cells of an organism, or organ- or tissue-specific, where the promoter is capable of preferentially initiating transcription in a particular organ, such as in the root, taproot, tuber, stem, leaf, petal, fruit, and seed. For example, a taproot-specific promoter is a promoter that is transcriptionally active predominantly in plant taproots (e.g., sweet potato SRD1 promoter), substantially to the exclusion of any other parts of a plant. Examples of root specific promoters include, but are not limited to RCc3 promoter, Arabidopsis PHT1 promoter, Arabidopsis Pyk10 promoter, LRX1 promoter, and PHT1 promoter. Examples of taproot specific promoters include, but are not limited to SRD1 promoter, Tlp promoter, His 1-r promoter and Mll promoter. Examples of tuber specific promoters include, but are not limited to a patatin class I promoter (B33-promoter), GBSS promoter, and lacasse promoter. Examples of fruit specific promoters include, but are not limited to tomato E8 promoter, tomato LA22CD07 promoter, and LesAffx.6852.2.S1_at promoter. Examples of seed specific promoters include, but are not limited to oleosin promoter, glycinin promoter, zein promoter, HaFAD2-1 promoter, HaAP10 promoter, phas-promoter, leB4-promoter, usp-promoter, and sbp-promoter. These and other promoters are known to those of ordinary skill in the art, and their use may be readily adapted to target Asnase expression in particular tissues or organs.

Inducible promoters have induced or increased transcription initiation in response to a stimulus, including but not limited to a chemical, environmental or physical stimulus, and stress (e.g., nitrogen depletion).

Expression constructs can include at least one marker gene, operably linked to a regulatory element (such as a promoter) that allows transformed cells containing the marker to be either recovered by negative selection (by inhibiting the growth of cells that do not contain the selectable marker gene), or by positive selection (by screening for the product encoded by the marker gene). Many commonly used selectable marker genes for plant transformation are known in the art, and include, for example, genes that code for enzymes that metabolically detoxify a selective chemical agent which may be an antibiotic or a herbicide, or genes that encode an altered target which is insensitive to the inhibitor. Several positive selection methods are known in the art, such as mannose selection. Alternatively, marker-less transformation can be used to obtain plants without any of the mentioned marker genes, the techniques for which are known in the art.

The transfer of foreign genes into the genome of a plant is called transformation. Transformation of plant species is a fairly routine technique. Advantageously, any of several transformation methods may be used to introduce the gene of interest into a suitable plant cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, and transformation using viruses, or pollen and microprojection.

Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. The plant is subsequently grown until the seeds of the treated plant are obtained. Methods for transforming plants are known to those having ordinary skill in the art. The nucleic acids or the construct to be expressed is preferably cloned into a vector, which is suitable for transforming Agrobacterium tumefaciens. Agrobacteria transformed by such a vector can then be used in a known manner for the transformation of plants, such as plants used as a model, like Arabidopsis, or crop plants, by way of example, tobacco and tomato plants, for example by immersing bruised leaves or chopped leaves in an agrobacterial solution and then culturing them in suitable media. The transformation of plants by means of Agrobacterium tumefaciens is well known in the art.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion are transformed and thus transgenic. Alternative methods are based on the repeated removal of the inflorescences and incubation of the excision site in the center of the rosette with transformed agrobacteria, whereby transformed seeds can likewise be obtained.

An especially effective method is the vacuum infiltration method with its modifications such as the “floral dip” method. In the case of vacuum infiltration of Arabidopsis, intact plants under reduced pressure are treated with an agrobacterial suspension, while in the case of the “floral dip” method the developing floral tissue is incubated briefly with a surfactant-treated agrobacterial suspension. A certain proportion of transgenic seeds are harvested in both cases, and these seeds can be distinguished from non-transgenic seeds by growing under the above-described selective conditions, or by selecting plants displaying a desired characteristic or phenotype. In addition, the stable transformation of plastids is advantageous because plastids are inherited maternally in most crops, thereby reducing or eliminating the risk of transgene flow through pollen.

The genetically modified plants and plant cells described herein can be regenerated via all methods with which the skilled worker is familiar.

Generally after transformation, plant cells or cell groupings are selected for the presence of the gene of interest. Wherein a marker gene was co-transferred with the gene of interest, plants may be selected by observing or detecting the marker gene, following which the transformed material is regenerated into a whole plant. To select transformed plants, the plant material obtained in the transformation may be subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. The transformed plants are screened for the presence of a selectable marker.

Alternatively, plants may be selected by observing whether the plants present with a phenotype indicative of the presence or absence of the gene of interest. As described herein, transformation of a plant with a nucleic acid sequence encoding an Asnase results in the reallocation of fixed carbon in the plant. Dependent on the promoter used, ectopic Asnase expression may result in for example, increased overall biomass, increased overall sugar, starch, and/or oil content, or increased sugar, starch, and/or oil content in a specific tissue or organ. Plants presenting with the desired phenotype may then be selected for regeneration, and used to generate lines homozygous for the transformed gene of interest.

Following DNA transfer and regeneration, putatively transformed plants may also be evaluated, for instance using Southern analysis, for the presence of the gene of interest, copy number and/or genomic organization. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then be further propagated through classical breeding techniques. The generated transformed organisms may take a variety of forms. For example, they may be chimeras of transformed cells and non-transformed cells, clonal transformants (e.g., all cells transformed to contain the expression cassette), grafts of transformed and untransformed tissues (e.g., in plants, a transformed rootstock grafted to an untransformed scion).

As used herein, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors described herein, all those constructions brought about by recombinant methods in which either (a) the nucleic acid sequences encoding proteins useful in the methods described herein, or (b) genetic control sequence(s) which is operably linked with the nucleic acid sequence described herein, for example a promoter, or (c) a) and b) are not located in their natural genetic environment or have been modified by recombinant methods, it being possible for the modification to take the form of, for example, a substitution, addition, deletion, inversion or insertion of one or more nucleotide residues.

The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods described herein, becomes a transgenic expression cassette when this expression cassette is modified by non-natural, synthetic (“artificial”) methods such as, for example, mutagenic treatment.

A transgenic plant is thus understood as meaning, as above, that the nucleic acids used in the methods described herein are not at their natural locus in the genome of the plant, it being possible for the nucleic acids to be expressed homologously or heterologously. However, as mentioned, transgenic also means that, while the nucleic acids described herein or used in the method herein are at their natural position in the genome of a plant, the sequence has been modified with regard to the natural sequence, and/or that the regulatory sequences of the natural sequences have been modified. Transgenic is preferably understood as meaning the expression of the nucleic acids described herein at an unnatural locus in the genome, i.e. homologous or, preferably, heterologous expression of the nucleic acids takes place. Preferred transgenic plants are mentioned herein.

In preferred embodiments, Asnase expression is increased or overexpressed in a target plant. As used herein, “increased expression” and “overexpression” refer to any form of expression that exceeds the original wild-type expression level. Methods for increasing expression of genes or gene products are well documented in the art and include, for example, increased expression of a gene or a gene homolog by transformation as described above, and overexpression driven by appropriate promoters, the use of transcription enhancers, or translation enhancers. Isolated nucleic acids which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution, or isolated promoters may be introduced into a plant cell in the proper orientation and distance from a gene of the present invention so as to control the expression of the gene.

Methods for Redirecting Fixed Carbon Allocation

Described herein are methods for redirecting a plant's allocation of fixed carbon toward carbohydrate polymers relative to a control plant. The methods described herein redirect fixed carbon allocation by promoting a decrease in overall plant protein content by limiting protein synthesis and induced degradation, resulting in the accumulation of non-nitrogenous compounds such as sugars, starch, cellulose, and oil. Generally, the methods comprise reducing cytoplasmic asparagines levels in a plant relative to a control. In certain embodiments, this is accomplished by increasing cytoplasmic L-asparaginase (Asnase) levels, resulting in hydrolysis of asparaginase. In a particular embodiment, the redirection of fixed carbon allocation occurs under non-stress conditions, wherein the plant is not limited in any particular nutrient, and in particular, nitrogen. In one embodiment, the method comprises increasing the expression of a nucleic acid encoding a polypeptide having at least 70% sequence identity to the soybean Asnase (L-asparaginase) polypeptide (SEQ ID NO: 2).

Increases in expression are relative to expression of the nucleic acid in a control plant. The choice of a suitable control plant is routine in experimental design and setup, and may include corresponding wildtype plants. The control plant is typically of the same plant species, and preferably, of the same variety as the plant to be assessed. “Control plant” refers not only to the whole plant, but also to plant parts. Referring to redirection of fixed carbon allocation relative to a control plant, it is meant that the target plant allocated higher ratios of carbon to non-nitrogenous carbonaceous compounds. This may refer to the entire plant, or a specific tissue or organ (e.g., taproot, fruit, and seed).

In certain embodiments, the nucleic acid encoding the polypeptide has a sequence identity to the soybean Asnase polypeptide selected from the group of at least 70%; at least 75%; at least 80%; at least 85%; at least 90%; at least 95%; at least 96%; at least 97%; at least 98%; at least 99%; and 100%. In a preferred embodiment, the nucleic acid encoding the polypeptide has a sequence identity to the soybean Asnase polypeptide of at least 95%. As used herein, “percent identity” describes the extent to which a nucleotide or polypeptide sequence are invariant throughout a window of alignment of sequences. Percent identity is calculated over the aligned length, preferably using a local alignment algorithm (e.g., BLASTn, BLASTp). The sequence identity values may be determined over the entire nucleic acid or amino acid sequence or over selected domains or conserved motif(s).

In one particular embodiment, the nucleic acid comprises the soybean nucleotide sequence (SEQ ID NO: 1) encoding the soybean Asnase. In another embodiment the nucleic acid comprises any nucleotide sequence that encodes the soybean Asnase polypeptide (SEQ ID NO: 2).

The expression of the nucleic acid may be increased in the plant by any means known in the art. For example, expression may be increased by introducing into the plants genome an exogenous promoter or enhancer element upstream of an endogenous Asnase gene, thereby increasing expression of the plant's endogenous Asnase gene. In a preferred embodiment, expression of Asnase is accomplished by introducing and expressing in the plant the nucleic acid encoding a polypeptide having at least 70% sequence identity to the soybean Asnase (L-asparaginase) polypeptide (SEQ ID NO: 2).

The nucleic acid may be operably linked to a promoter. In certain embodiments, the promoter is a ubiquitous constitutive promoter (e.g., cauliflower mosaic virus (CaMV) 35s promoter). Wherein the promoter is a ubiquitous constitutive promoter, increased expression of the nucleic acid occur throughout most, if not all, plant tissues. The promoter may also be a tissue specific promoter, which results in increased expression of the nucleic acid in a particular tissue or organ. Tissue specific promoters may be selected to target increased expression of the nucleic acid in nearly any plant tissue such as, for example, root, taproot, tuber, stem, leaf, petal, fruit, and seed. Many such tissue specific promoters are known in the art, and one having ordinary skill in the art will be able to recognize and select a promoter which may be used to drive increased expression of the nucleic acid in a target tissue or organ. By way of example, tissue specific promoters useful in the methods described herein include the patatin tuber specific promoter, the E8 tomato fruit specific promoter, the SRD1 taproot specific promoter, the Mll taproot specific promoter, the His 1-r taproot specific promoter, the Tlp taproot specific promoter, the oleosin seed specific promoter, and the glycinin seed specific promoter.

In certain aspects wherein the promoter is a ubiquitous constitutive promoter, whole-plant biomass is increased. This is a result of increased production of carbonaceous solids throughout the entire plant, including sugars, starch, and oils, and in particular, cellulose. The methods describe herein can increase the cellulose content of a plant, as measured per unit mass, 20-50% that of a wild-type control plant. Ubiquitous constitutive promoters also reduce free asparagines in the plant by up to 75%, and increase sugar content 2-3 fold relative to a control plant. Increasing these carbonaceous solids throughout the plant provides greater overall biomass using the same resources as the control plant. Improved resource utilization, such as fixation of carbon in non-nitrogenous compounds rather than plant proteins improves plant efficiency by “making more from less.” Furthermore, increasing expression of the nucleic acids described herein can accelerate a plant's lifecycle by 5-20%. This is particularly advantageous in that it significantly increases double-cropping potential of many different varieties of plants.

Wherein the promoter is a tissue or organ specific promoter, sugars, starch, cellulose, and/or oil content may be increased in a target tissue or organ of the plant relative to the tissue or organ of a control plant. Sugar content may be increased specifically, for example, in the tomato fruit, corn kernel, and soybean bean. These increases may be desirable, in the cases of tomato and corn, for enhanced sweetness of tomatoes and corn for consumption, while a sweeter soybean bean may be similarly desirable by humans, or have increased value as feed. Oil content of many oil crops may be similarly improved by targeting increases in Asnase expression to oil-producing tissues and organs of crops, including but not limited to soybean, cotton, sunflower, canola and rapeseed), peanut, sesame, flax, safflower, and Camelina spp. In such crops, even small increases in oil content are economically valuable. Starch and sugar content of taproots and tubers may also be increased by methods described herein, for example, carrot, sugar beet, cassava, potato, yam, and sweet potato.

Redirection of a plant's allocation of fixed carbon toward carbohydrate polymers may, as discussed above, be ubiquitous or tissue specific. In certain embodiments, however, expression of the nucleotide encoding Asnase may be increased both ubiquitously and in a particular tissue or organ. By expressing two copies of the nucleotide in the cell, each under a different promoter, the nucleic acid may be increased generally throughout the plant, as well as in a particular tissue or organ. Therefore, in certain embodiments the method further comprises introducing and expressing a second copy of the nucleic acid in the plant, wherein the second copy is operably linked to a second promoter that is different from the promoter linked to the first copy of the nucleic acid. The two copies of the nucleic acid under the control of two different promoters allows for the generation of plants having increased growth rates and accelerated lifecycles, and tissues or organs (e.g., taproot, tuber, seed) with higher levels of sugar, starch, and/or oil relative to control plants.

The economic value of increasing carbon fixation in non-nitrogenous compounds in whole plants or specific tissues and organs of the crops described herein, as well as other crops, will be evident to those of ordinary skill in the art.

Constructs

Describe herein are constructs useful for generating transgenic plants having altered carbon allocation, wherein the transgenic plant has a higher C:N ratio than a control plant due to reallocation of carbon from nitrogen-rich proteins to non nitrogenous carbohydrates including sugars, starch, cellulose, and oils. Constructs comprise a nucleic acid encoding a polypeptide having at least 70% sequence identity to the amino acid sequence of soybean Asnase (SEQ ID NO: 2), and one or more control sequences, such as a promoter, capable of driving expression of the nucleic acid in a plant cell. Optionally, the construct comprises a transcription termination sequence. A termination sequence is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

Transgenic plants comprising the construct may be generated by transforming a plant, plant part, or plant cell with the construct described herein and cultivating the transformed plant, plant part, or plant cell to produce a transgenic plant. Plants comprising the construct can thereafter be selected and used to generate homozygous lines. Transgenic plants comprising the construct will preferably have altered fixed carbon allocation relative to control plants, wherein the transgenic plant or tissues or organs thereof have higher C:N ratios than those seen in the control plants.

The nucleic acid of the construct is one that encodes an Asnase polypeptide, and is the same as that described and discussed above. The amino acid sequence encoded by the nucleic acid forms an Asnase capable of hydrolyzing asparagine. In certain embodiments, the nucleic acid encoding the polypeptide sequence has a sequence identity to the Asnase amino acid sequence of SEQ ID NO: 2 selected from the group consisting of at least 70%, at least 75%, at least 80%, at least 85%, at least 90%, at least 95%, at least 96%, at least 97%, at least 98%, at least 99%, and 100%. In other embodiments, the nucleic acid comprises the soybean nucleotide sequence (SEQ ID NO: 1) encoding the soybean Asnase. In yet another embodiment, the nucleic acid comprises any nucleotide sequence that encodes the soybean Asnase polypeptide (SEQ ID NO: 2).

The construct may further comprise a second copy of the nucleic acid, and one or more control sequences capable of driving expression of the second copy of the nucleic acid sequence. Wherein the construct comprises two copies of the same nucleic acid sequence, the control sequences driving their expression are different. For example, a ubiquitous constitutive promoter drives expression of one copy of the nucleic acid sequence, while a tissue specific promoter drives expression of the other copy. Ubiquitous constitutive promoters and tissue or organ specific promoters useful in the constructs are the same as those described above.

The construct may be used as a simple promoter-gene construct for use in transformation by methods known in the art, but preferably further comprises a vector suitable for plant transformation. Such vectors are well known in the art and may be identified and selected by those having ordinary skill in the art.

Plants, plant parts, or plant cells comprising a construct describe herein are also contemplated. Many plants may be transformed to comprise the construct described herein, including but not limited to soybean, potato, tomato, tobacco, Camelina spp., maize, carrot, switchgrass, sugar beet, cassava, sweet potato, yam, Brachypodium, onion, safflower, sunflower, canola (rapeseed), hemp, cotton, sesame, peanut, flax, rice, wheat, and oats. Other plants, including trees, may also be transformed using the construct described herein. From the disclosure and examples herein, it will be recognized that additional plant varieties may be transformed with the construct. This includes most any plant that would benefit from reduced cytosolic levels of asparagine, either in the whole plant, or a specific tissue or organ, ultimately resulting in a higher C:N ratio in those cells, tissues, or organs where asparagine levels are reduced.

Harvestable parts of plants comprising the construct described herein are also within the scope of the present disclosure, wherein the harvestable parts are, for example, shoot biomass, fruits, roots, taproots, and seeds.

Methods for Producing Transgenic Plants

Any known method known in the art may be used to make a transgenic plant described herein. Generally, the method comprises introducing and expressing in a plant or plant cell a nucleic acid encoding a polypeptide having at least 70% sequence identity to the amino acid sequence of soybean Asnase (SEQ ID NO: 2), and cultivating the plant or plant cell under conditions promoting plant growth and development. Such conditions will vary, dependant on the variety of transgenic plant to be produced. Proper conditions for promoting plant growth and development for a particular plant variety will be discernible to one of ordinary skill in the art.

In certain embodiments, the nucleic acid has a sequence identity to the amino acid sequence of soybean Asnase of at least 70%, at least 75%, at least 80%, at least 85%, at least 90%, at least 95%, at least 96%, at least 97%, at least 98%, at least 99%, and 100%. In a preferred embodiment, the nucleic acid has a sequence identity to the amino acid sequence of soybean Asnase of at least 95%. In another embodiment, the nucleic acid comprises the nucleotide sequence of SEQ ID NO: 1 or encodes a polypeptide comprising the amino acid sequence of SEQ ID NO: 2.

Transgenic plants, plant parts, harvestable parts of such plants, and products obtained from such plants are also contemplated herein. Harvestable plant parts of transgenic plants include, for example, shoot biomass, fruits, taproots, and seeds. Such harvestable parts may be utilized in the as food, animal feed, and production of biofuels. Products obtained from the transgenic plants include oils and other food products, biofuel biomass, biofuel, fodder, timber, Christmas trees, paper pulp, mulch, and fiber . . . . The advantages of transgenic plants having higher sugar, starch, cellulose, and/or oil content relative to control plants will be readily apparent to those of skill in the art. By way of illustrative example only, even slight increases in oil content of soybean (1-2%) would be economically valuable. Examples of plants in which nucleic acids of the present disclosure may be introduced and expressed are described above.

Increasing Oil Production in Algae

Of all clean energy options in development, it is algae-based biofuel that most closely resembles the composition of crude oil. Many strains of microalgae are efficient producers of triacylglycerols, which can be converted for use as biodiesel. Algae have been identified as a viable feedstock for biofuels due to their efficient abilities to convert sunlight and CO₂ to biomass, synthesize large quantities of lipids (20-75% dry mass), thrive in saline water, grow on non-arable land, and grow in open or closed systems. Microalgae are considered to be superior oil-producers compared to terrestrial competitors (e.g., corn, palm, rapeseed, jatropha, and soybean) because microalgae devote fewer resources to the synthesis of structural components such as cellulose and lignin. While nitrogen-deficient conditions lead to an increase in lipid/cell, there is an overall decrease in the growth and cell-mass produced, similarly to plants, as described above. There is a need for methods to increase oil production that are economically competitive and sustainable.

Methods are described herein for increasing oil production in algae, comprising increasing expression in algae a nucleic acid encoding a polynucleotide having at least 70% sequence identity to the soybean Asnase (L-asparaginase) polypeptide (SEQ ID NO: 2). Increases in Asnase result in a decrease in asparagines, thereby redirecting carbon fixation toward oils, similarly to plants. In certain embodiments, this redirection of fixed carbon allocation occurs under non-stress conditions, wherein the algae are not limited in any particular nutrient, and in particular, nitrogen. These methods circumvent the issues of decreased growth and cell-mass seen with nitrogen starvation. Increases in expression are relative to expression of the nucleic acid in control algae.

In certain embodiments, the nucleic acid encoding the polypeptide has a sequence identity to the soybean Asnase polypeptide selected from the group of at least 70%; at least 75%; at least 80%; at least 85%; at least 90%; at least 95%; at least 96%; at least 97%; at least 98%; at least 99%; and 100%. In a preferred embodiment, the nucleic acid encoding the polypeptide has a sequence identity to the soybean Asnase polypeptide of at least 95%. In a particular embodiment, the nucleic acid comprises the soybean nucleotide sequence (SEQ ID NO: 1) encoding the soybean Asnase. In another embodiment the nucleic acid comprises any nucleotide sequence that encodes the soybean Asnase polypeptide (SEQ ID NO: 2).

Increasing expression of the nucleic acid may be achieved by introducing and expressing the nucleic acid in algae. Any method known in the art for transforming algae with the nucleic acid, including but not limited to glass bead-assisted transformation and biolistic transformation. The nucleic acid can be operably linked to a promoter capable of driving expression of the nucleic acid in algae. Such promoters are known in the art and include, for example, CaMV 35s and SV40 promoters. Other promoters are known in the art, and may work best with particular algal species. For a review of useful promoters for driving expression of exogenous nucleic acids in algae, and methods for transforming algal cells (including selection methods), see Hallmann (2007), Transgenic Plant Journal, 1(1):81.

Reallocating carbon fixation by the methods describe herein result in increased oil production, enhanced growth rates, and accelerated life cycle relative to a control. Products obtained from the algae, such as oils and biomass.

The algae produced by methods described herein may also be used to scavenge environmental CO₂. By enhancing growth rates and causing increased carbon fixation, the resulting algae can have enhanced CO₂ scavenging capabilities.

### Examples

The methods and embodiments described herein are further defined in the following Examples. Certain embodiments of the present invention are defined in the Examples herein. It should be understood that these Examples, while indicating preferred embodiments of the invention, are given by way of illustration only. From the discussion herein and these Examples, one skilled in the art can ascertain the essential characteristics of this invention and without departing from the spirit and scope thereof, can make various changes and modifications of the invention to adapt it to various usages and conditions.

### Example 1

### Materials and Methods

Asnase Expression Construct.

The following constructs were designed and produced for use in potatoes. The same or similar constructs may be used in other crop varieties. Changes in promoter allow for tissue-specific expression of Asnase in a target plant. Coding sequences for asparaginase from other plant varieties may also be used. From the disclosure herein, one of ordinary skill in the art will be able to adapt the construct for a desired purpose.

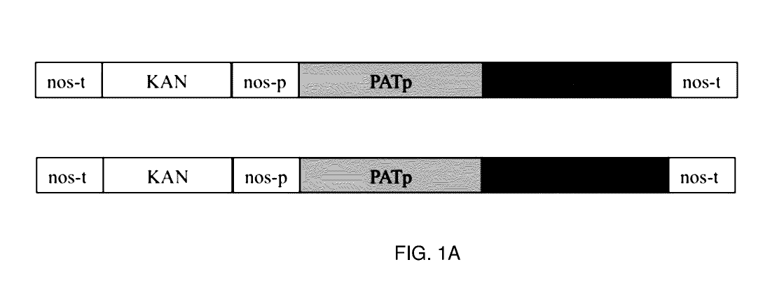

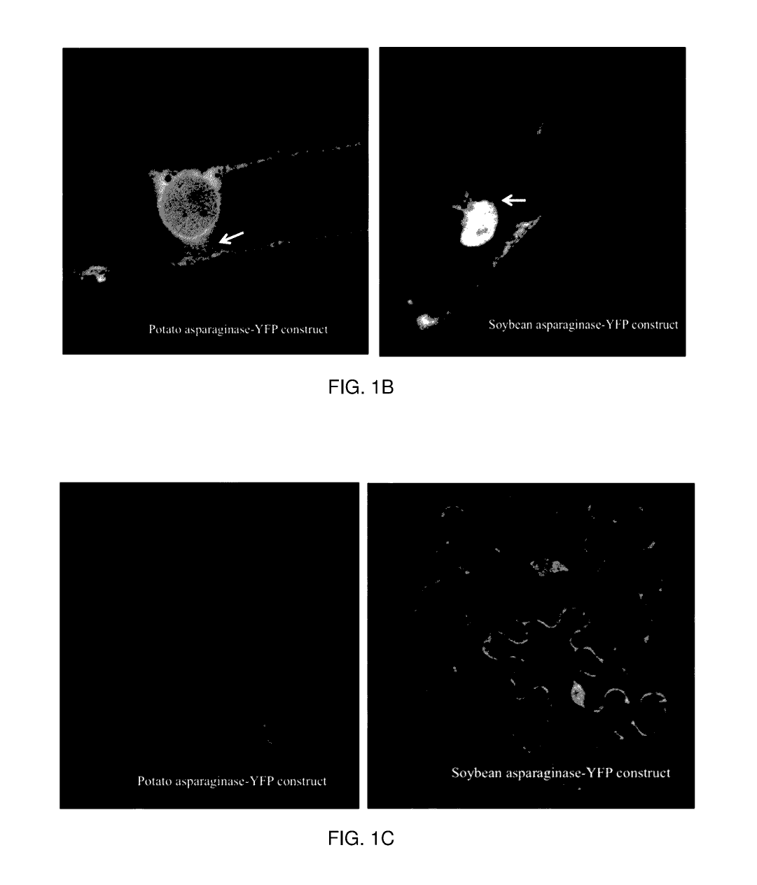

The coding regions for both the potato and soybean asparaginase (Asnase) genes were cloned by using PCR to amplify the region from cDNA derived from RNA isolated from potato tubers and immature soybean (150 mg) cotyledons, respectively. Total RNA was isolated using Trizol and then used as a template for cDNA by SuperscriptII First Strand Synthesis System (Invitrogen) using oligo T primer according to manufacturer's instructions. PCR was then performing using specific Asnase primers, for either potato Asnase or for soybean Asnase. Asnase PCR-amplified fragments were cloned into TOPO™ vector and subsequently digested to liberate the Asnase fragments. The potato and soybean Asnase coding regions were then separately ligated into pMON999 (Monsanto Co., St. Louis Mo.), resulting in the Asnase cDNAs being directed by the CaMV 35S promoter and NOS 3′ end sequence. The CaMV 35S promoter was subsequently removed from the plasmids by digestion and replaced with patatin promoter. The patatin promoter was used to obtain tuber-specific high expression of Asnase in potato. Chimeric gene cassettes were excised from the intermediate plasmids and genes were ligated into the binary vector with this plasmid also containing the neomycin phosphotransferease II (nptII) gene for selection of transformed plants. Schematic representations of constructs containing Asnase-coding sequence from potato (pPotASN) and soybean (pSoyASN) are shown in FIG. 1A. The binary vectors containing pPotASN or pSoyASN were electroporated into Agrobacterium tumefaciens GV2260.

Production of Transgenic Plants.

The methods of carbon re-allocation in crops use similar approaches, applicable to potato, maize, tomato, soybean, tobacco, and Camelina spp. Each of these crop plants was chosen for their capacity to be transformed, the genetic infrastructure available, and their potential value as products. The methods are similarly applicable to other crop plants. The production and analysis of transgenic plants uses a variety of methods including Agrobacterium-mediated transformation of various plant organs and particle bombardment of tissues and cultures. Transformations are performed and plants are selected to produce homozygous lines. As an example, methods for potato transformation and selection are now described.

Potato (Solanum tuberosum L. var. Atlantic) shoot cultures were obtained (in vitro potato collection, Cornell University) and subsequently grown in vitro on MS basal medium containing 2% sucrose and no phytohormones. Agrobacterium-mediated transformation of potato stem explants were performed. From 4 wk old in vitro grown plants, the internodal stem segments (3-5 mm) were incubated for 15 min in an overnight OD₆₀₀ 0.8-1.0 culture of Agrobacterium tumefaciens containing one of the Asnase expression plasmids (pPotAsnase or pSoyAsnase), blotted dry on sterile Whatman paper, and transferred to callus induction medium (CIM) for 4 wks. The CIM is a MS basal medium containing 0.1 mg/liter naphthaleneacetic acid and 2.5 mg/L zeatin riboside. Explants were transferred to shoot-inducing media, MS basal medium containing 0.3 mg/L gibberellic acid and 2.5 mg/L zeatin riboside. Shoots formed within 4 wks were individually transplanted to root inducing media, MS basal media with no phytohoromes. All tissue culture media, callus, shoot and root inducing media contained 100 mg/L kanamycin for transgenic tissue selection and 150 mg/L cefotaximine to deter Agrobacterium growth. All cultures were incubated at 20±2° C. in a 16-h photoperiod.

The presence of the transgene was determined by genomic PCR using primers specific for the nptII selectable marker plant gene and DNA was isolated from putative transgenic leaf tissue. All PCR kanamycin positive plants were moved to the greenhouse and tubers harvested after 4 months of growth. Over 20 lines were produced for each construct. The positive tubers were collected and regrown as clones for additional four month growth cycles to obtain second and onward generations of the positive lines.

Test Crops and Compositional Alterations.

Biotechnology engineering methods utilizing the nucleic acids described herein induce a plant to reprogram its growth and metabolism to favor high carbon content, growth, and substance accumulation. For some crop plants, where the goal is to decrease life-cycle time and to increase carbonaceous output traits, this single strategy is an effective means to enhance crop efficiency.

Protein Assessment and Proteomics

Protein composition of tissues and organs were screened using 2D IEF SDS/PAGE with differential spot distribution documented between wild type and transgenics as well with nitrogen input changes. Soluble protein was extracted from ˜250 mg of tissue of transgenic tubers in 0.6 ml of extraction buffer (0.1 M Tris-HCL pH8.8, 10 mM EDTA, 0.4% (v/v) 2-mercaptoethanol, 0.9M sucrose, 5 mM PMSF). Protein pellets were solubilized in Destreak rehydration solution (GE Healthcare, Pillsburgh Pa.) and 150 μg total protein was then loaded onto an immobilized pH gradient (IPG) strip (11 cm, pH 4-7; BioRad) and hydrated overnight. Isoelectric focusing occurred for a total of 40 kVh on a Protean IEF Cell (Biorad) and then the second dimension performed on an SDS-PAGE 8-16% linear gradient gel run on a criterion gel apparatus for 15 min at 60 V and then 1 hr at 200 V. Gels were stained overnight in 0.1% (w/v) coomassie brilliant blue R-250 in 40% methanol/10% acetic acid (v/v) and then de-stained 3 times in 40% methanol/10% acetic acid (v/v).

Protein identification was by tryptic digestion followed by LC-MS/MS analysis using MudPit approach (e.g., Delehunty and Yates (2007), Biotechniques, 43:563). Proteomics assessment is performed to study changes in plant phenotype (e.g., Herman et al. (2006), J. Exp. Bot., 57:1; Schmidt and Herman (2008), Plant Biotechnology, 6:832).

Compositional and Systems Biology Analysis of Phenotype.

For each crop, the effects of decreased nitrogen-status is evaluated. In grass plants over-expressing Asnase, a series of compositional and morphological assays demonstrated that targeted changes induced a shift in composition favoring the accumulation of carbohydrate polymers. These assays establish compositional changes in output phenotype with regard to reduced nitrogen content (amino acid, protein, chlorophyll) and increased carbohydrate content (free sugars, starch and cellulose polymers).

Metabolite Profiles Including Amino Acids and Carbohydrate Compositional Analysis.

Non-targeted metabolomic analysis of transgenics using LC and GC methodology (Allwood et al. (2011), Methods Enzymol., 500:299) was performed. Extractions of transgenic and wild-type tissues were performed and analyzed. The analysis used statistical and Bio-informatic analysis. Differences between the wild-type and transgenics were characterized for each specific chemical species, analyzed with regard to pathway changes and as changes in principal component. Carbohydrate analysis and composition is performed at the USDA Carbohydrate Center in Athens, Ga.

### Example 2

### Cytosolic Localization of Asnase and Metabolic Changes Resulting from Increased Asnase

Fusion cassettes with both the potato and soybean Asnase open reading frames were produced by digesting and moving the open reading frames into the vector, placing the Asnase coding regions in frame with yellow fluorescent protein (YFP) behind the enhanced 35S CaMV promoter. Onion epidermal cell layers were obtained from fresh onions and bombarded with fluorescent protein (YFP) behind the enhanced 35S CaMVp. Post-bombardment tissue was kept moist in dark conditions and monitored periodically for YFP expression. Expression was seen as early as 4 hr post-bombardment and continued for the following 3-4 days.

Agroinfiltration of fully expanded tobacco (Nicotiana tabacum Samsung) leaves in planta was performed using the YFP fusion cassettes in Agrobaterium tumefaciens LBA4404. Tobacco tissues were infiltrated with a syringe into intercellular spaces. Infiltrated tissues were covered and the plants were placed back into the greenhouse overnight with conditions of 25° C. and 16 hr photoperiod.

For both onion epidermal cells and tobacco leaf tissue, areas exhibiting transient YFP expression were first detected by the use of a fluorescent dissecting scope using mercury lamp excitation and BP 460-500 nm excitation filter, 505 dichroic, and 510 LP emission filter. Positive expression areas were then examined in detail using a Zeiss LSM510 confocal microscope with excitation using the 514 nm line of a argon gas laser, and aBP 535-590 nm nm emission filter.

Both the tobacco and onion cells expressed the Asnase-YFP fusion protein after a short rest/recovery time. The onion-skin cells bombarded with the either the potato or soybean Asnase-YFP construct exhibited fluorescence in single fluorescent cells that were easily visualized in black background low magnification fields. Cells were visualized under high magnification of optical sections to optimize visualization and demonstrated that YFP fluorescence was cytosolic with both the potato and soybean Asnase-YFP proteins. The intracellular fluorescence within the onion cells delineates the central vacuole with cytoplasmic filaments connecting cytoplasm domains (FIG. 1B; arrows), showing that both potato- and soybean Asnase-YFP were localized in the cytoplasm. Numerous dark bodies are embedded in the fluorescent cytoplasm that is consistent with these being organelles such as mitochondria, peroxisomes, and proplastids excluding the fluorescent fusion protein. Parallel experiments infiltrating potato or soybean Asnase-YFP constructs into tobacco leaves mediated by Agrobacterium yield similar results. In infiltrated tobacco leaves most/all of the observed cells expressed the YFP fusion protein with the fluorescence of either the potato or soybean-YFP restricted to the peripheral located cytoplasm surrounding the large central vacuole (FIG. 1C).

To test the effect of systemic destruction of free Asn in tobacco, the soybean Asnase gene, regulated by 35S, was transferred to tobacco. Transgenic plants were selected, and subjected to recurrent selection to obtain homozygous lines.

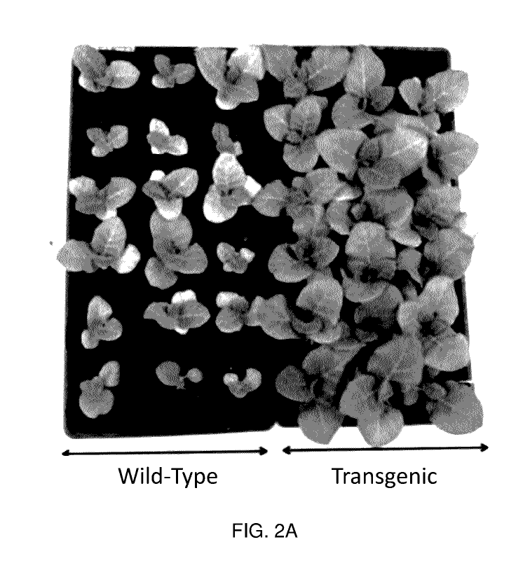

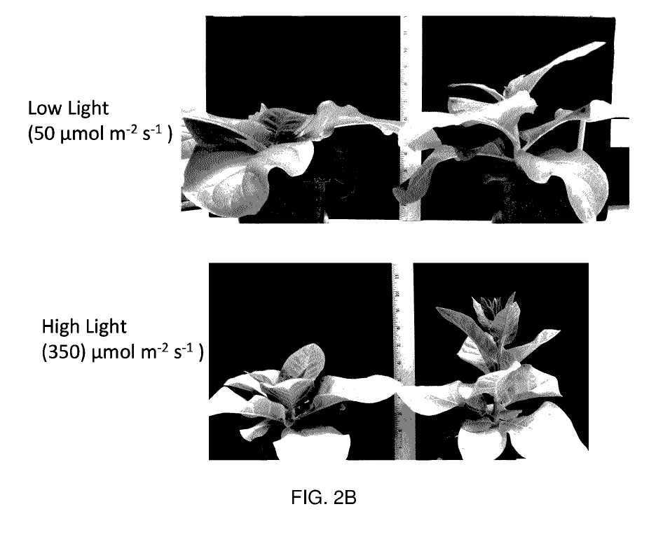

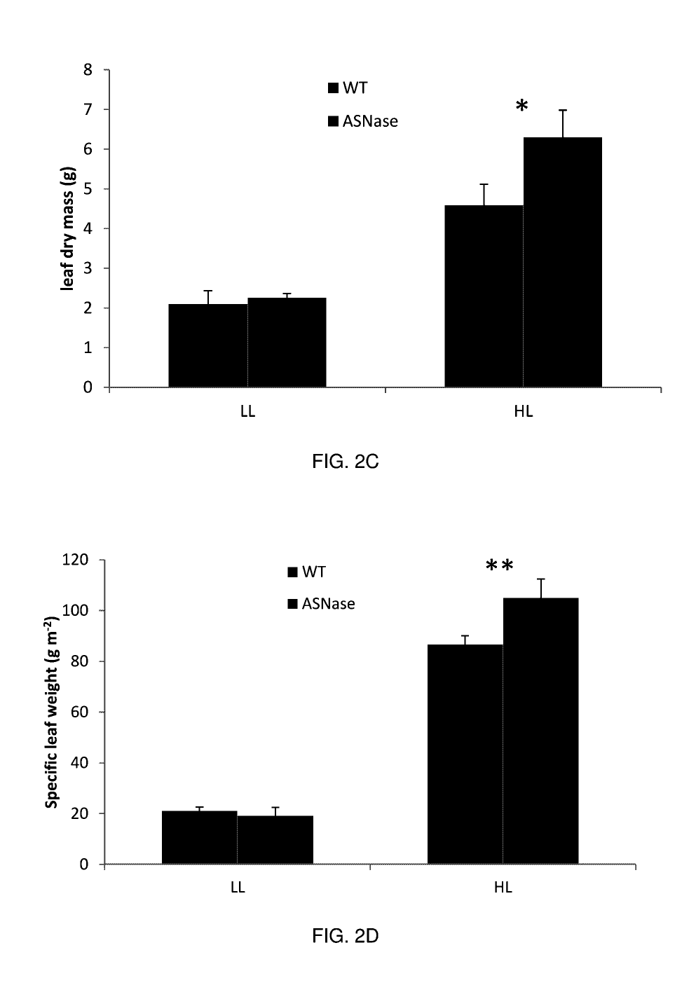

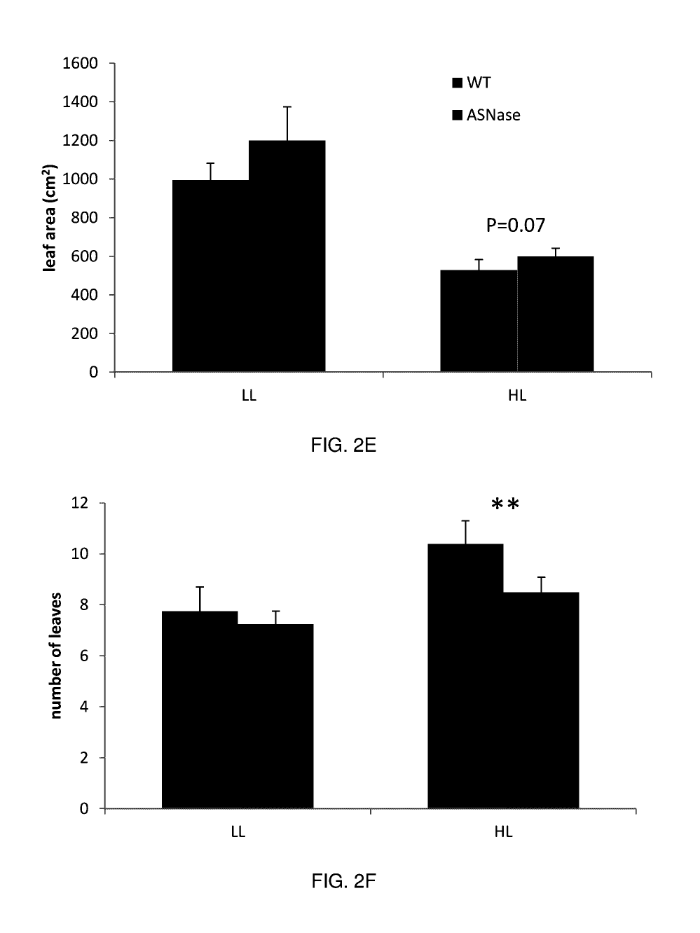

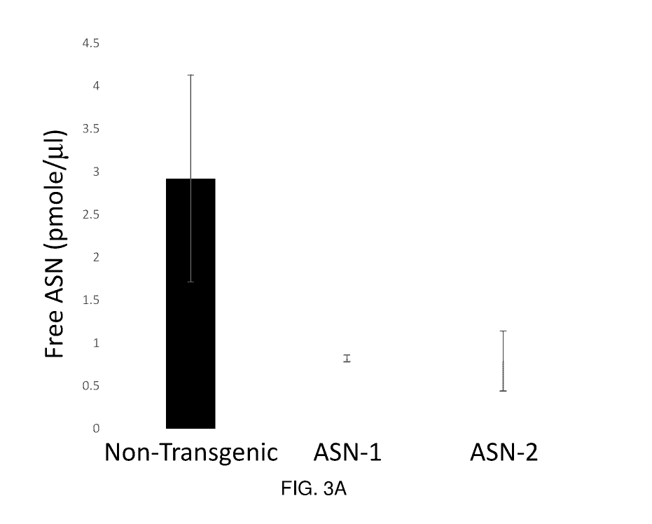

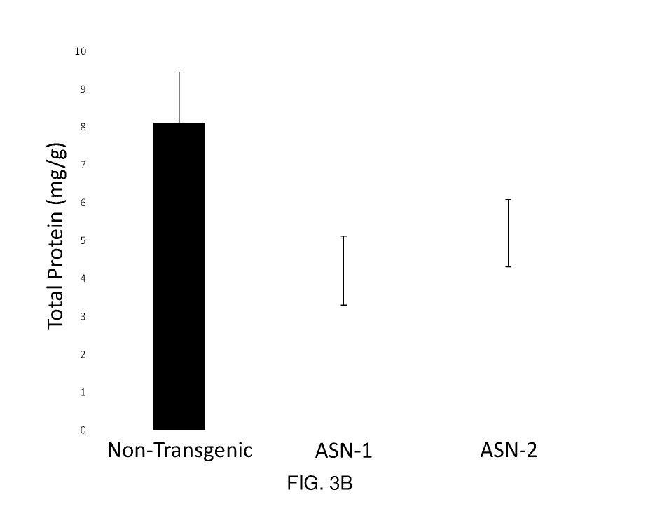

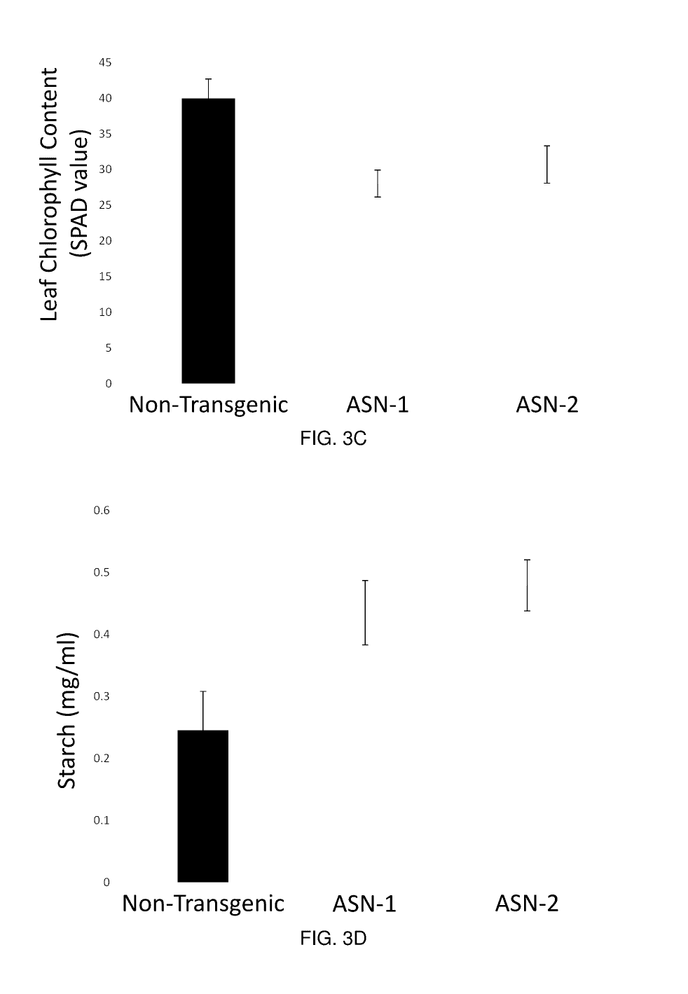

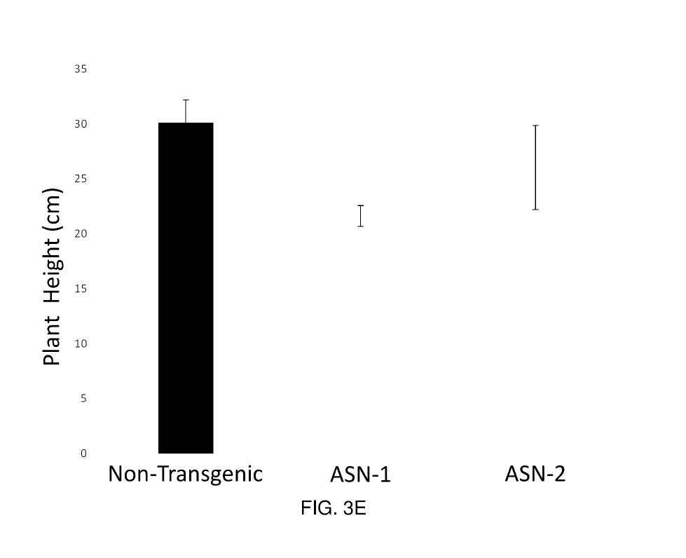

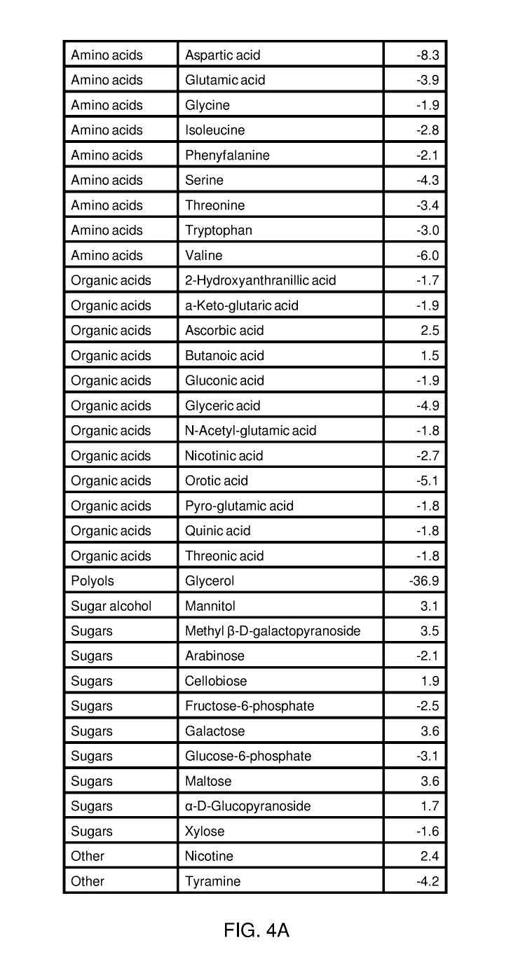

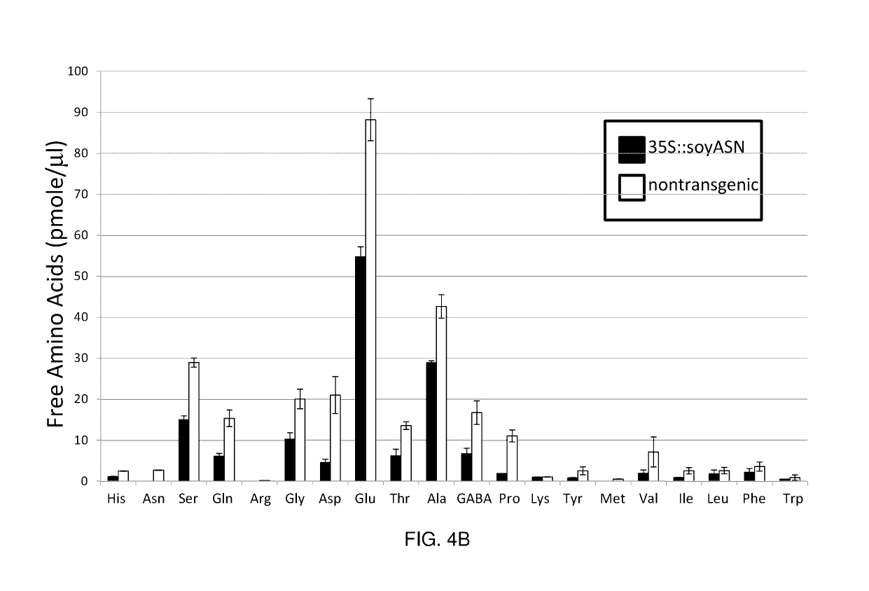

The ectopic expression of Asnase in the tobacco model shifts composition and enhances growth. The ectopic Asnase expressing plants appeared overtly normal with the notable changes in the accelerated growth that rapidly outstripped the control. While the leaf morphology was normal, the leaves of the ectopic Asnase expressing plants were larger (FIG. 2). Using amino acid analysis as well as non-targeted metabolomics assays, overall reduction of free Asn averaged 75% (FIGS. 3A and 4B).

Tobacco leaves expressing ectopic Asnase exhibit a reduction in both protein (FIG. 3B) and chlorophyll (FIG. 3C) content (measured as SPAD), and about two-fold greater starch accumulation (FIG. 3D). To some degree, these overt characteristics are the inverse of the consequences of ectopic asparagine synthetase.

While the Asnase expression could be thought to be another variation of inducing nitrogen limitation using an intrinsic rather than extrinsic approach, however, on further detailed analysis, systemic hydrolysis of Asn results in a complex trait that has potential for development into enhanced growth and compositional traits.

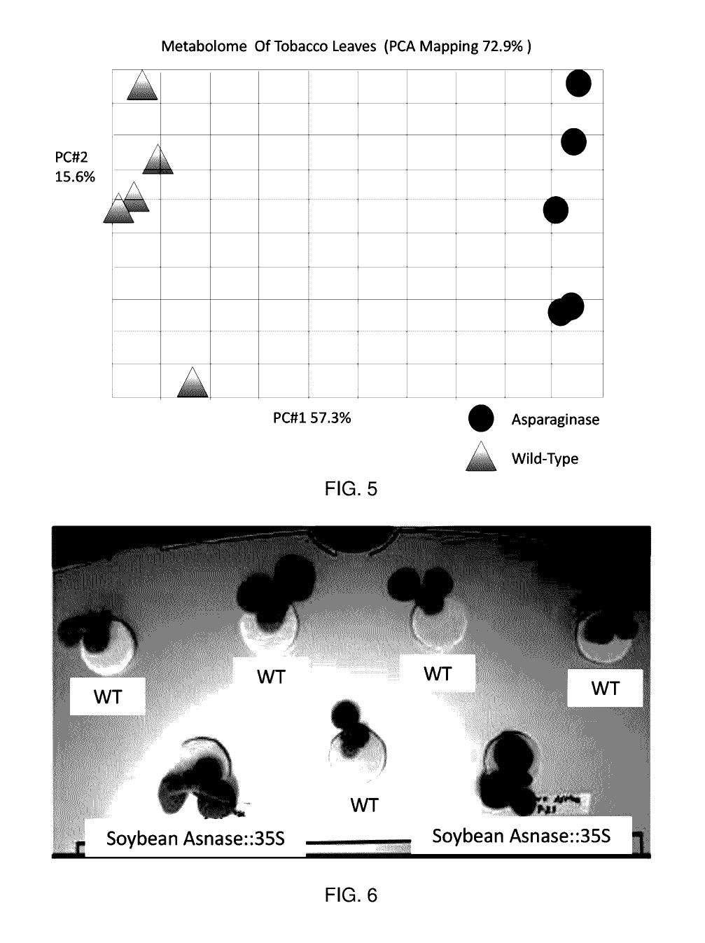

To further analyze the metabolic consequences of free Asn reduction, non-targeted metabolomics assessment assays were performed on one of the insertion events. Results are shown in FIG. 4A, with a principle component summary shown in FIG. 5. The metabolomics results showed a net reduction in free amino acids and an increase in some sugars, indicative of a reconfiguration of the tobacco plant's metabolism shifting toward accumulation of carbonaceous polymers that do not contain nitrogen.

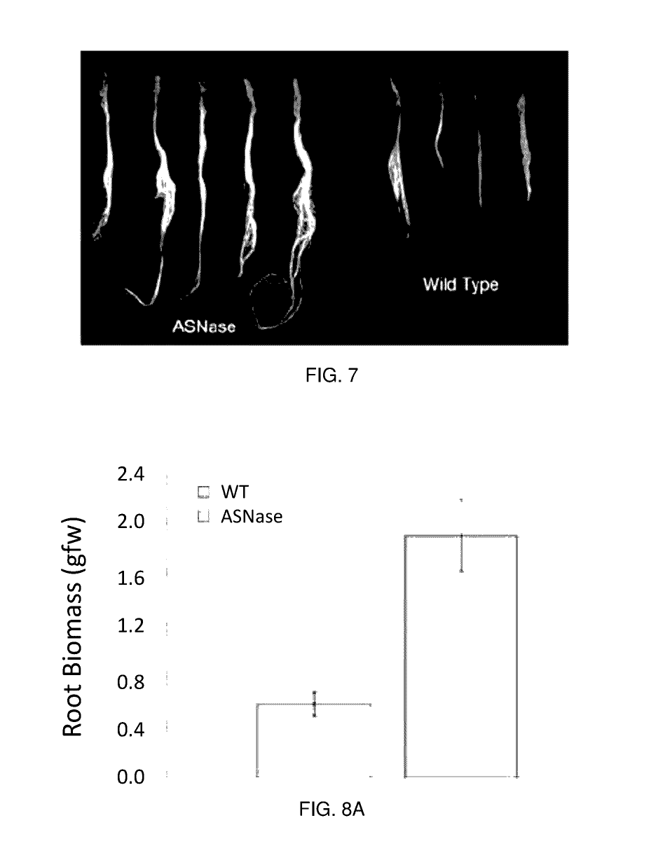

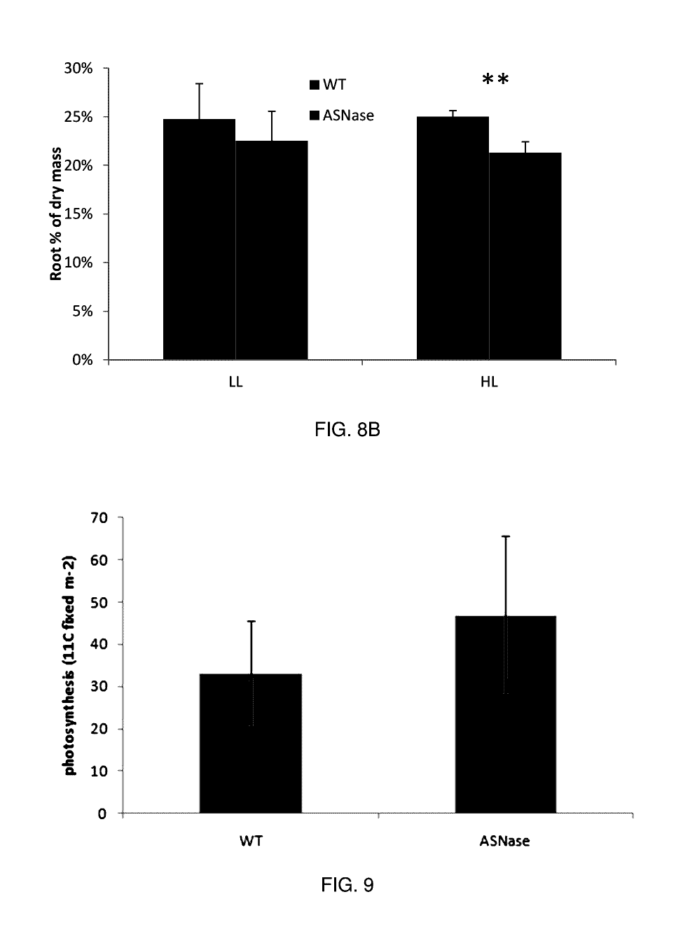

To further analyze the carbon flux consequences of Asnase expression, metabolic flux analysis and supporting assays were performed on hydroponic-grown plants of the same insertion event used for the non-targeted flux analysis. Experiments were performed using short-lived ¹¹C isotope input as a photosynthetic carbon label to discern the differences in carbon flux (Best et al. (2011), Carbohydrate Res., 346: 595; Hanik et al. (2010), J. Chem. Ecol., 36: 1058) and allocation of the Asnase expressing plants in comparison to wildtype. Hydroponic-grown plants were used in preference to soil-grown plants for reasons of experimental design. As with soil-grown plants, the hydroponic-grown plants exhibit a higher growth rate (FIG. 6) and more extensive root proliferation (FIG. 7), which was two-fold greater in mass than wild-type root mass (FIG. 8A).

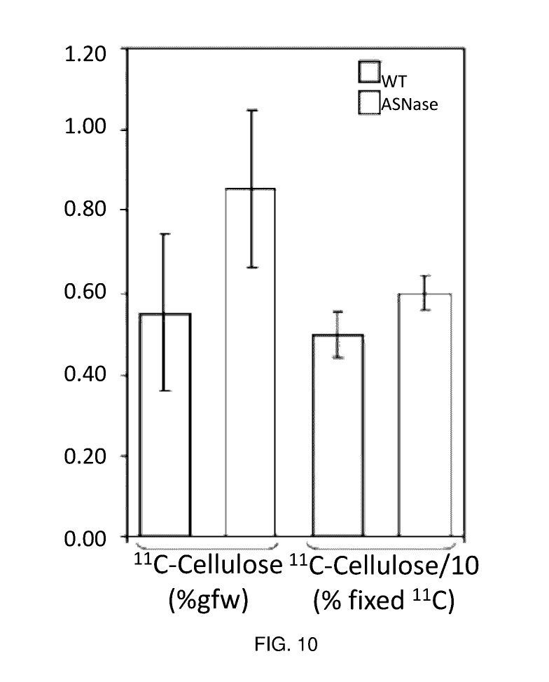

Carbon flux and allocation experiments were also performed using ¹¹C carbonate labeling (Ferrieri et al (2004), Plant Cell Environ., 25: 591). The results of these experiments show that plants expressing ectopic Asnase have increased capacity to assimilate ¹¹C into biomass. The overall capacity of the Asnase expressing plants to fix ¹¹C was greater than the wild-type plants grown in parallel (FIG. 9). The greater physical mass of the Asnase-expressing plants required increased carbon allocation into supporting structural elements, in particular cell walls. Cellulose compositional assessment of the wild-type and Asnase-expressing plants showed that overall cellulose content, per unit mass, was about 35% greater in the Asnase-expressing plants (FIG. 10). This demonstrated the correlation that increases in C fixation lead to a parallel increase in cellulose content, resulting in shifting the plant's biomass composition to glycan polymers, and in particular, starch and cellulose. Using ¹¹C flux analysis of carbon fixed into cellulose, the overall rate of carbon flow into cellulose of the Asnase-expressing plants was about 20% greater than the wild-type. The data showed that by reducing free Asn, the plants reconfigured their metabolism and resulting in carbon accumulation to favor carbonaceous polymers (cellulose and starch) and free sugars, their immediate precursor.

### Example 3

### Reduction of Free Asn in Potato Tuber Shows Asn Reduction can be Targeted to be an Organ-Specific Trait

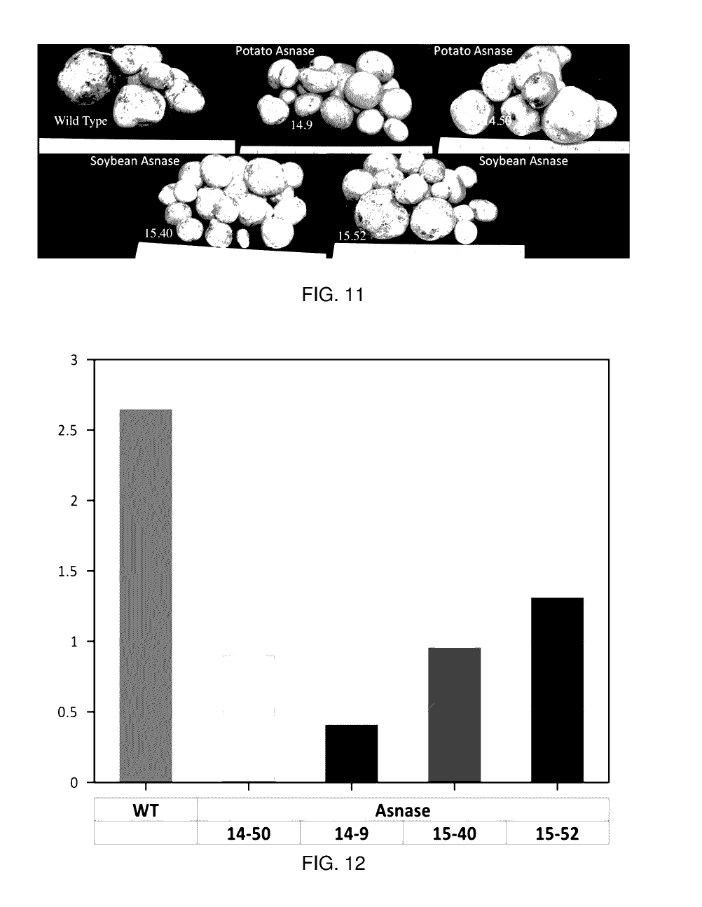

Transgenic potatoes were produced to test whether potatoes expressing ectopic Asnase could achieve decreased Asn levels. To accomplish this, both soybean and potato Asnase were ectopically expressed in potato tubers under the control of the patatin tuber-specific promoter. The resulting Asnase over-expressing potatoes exhibited 55-75% reduction in free Asn in the tubers, while the potato's plant growth, overt productivity, as well as size of the potatoes produced, was similar to controls (FIG. 11).

As with tobacco, the potato results showed enhanced crop value from reducing free Asn. The results showed that compositional changes can be induced by causing plants to rebalance their carbon allocation, resulting in accelerated vegetative and root growth and shifting the allocation of carbon to carbonaceous output traits. Refining this engineering strategy with combinations of regulatory elements and crops is a valuable method to enhance the value traits in crops.

An Increase in Asparaginase Activity in Potato Demonstrated Tuber-Specific Decrease in Asparagine and Increased Free Sugar Production.

The tubers of transgenic potato plants were screened for ectopic Asnase expression using RT-PCR with primers for the transgene in concert with genomic PCR with primers specific for the kanamycin gene. Four lines (two lines contain the soybean gene, and two lines contained the potato gene) from among the numerous transgenic plant lines were chosen for further analysis. Each line selected exhibited high levels of expression of the transgene. RT-PCR was used as a screening tool to assess relative steady state transcript abundance and to choose lines for further analysis.

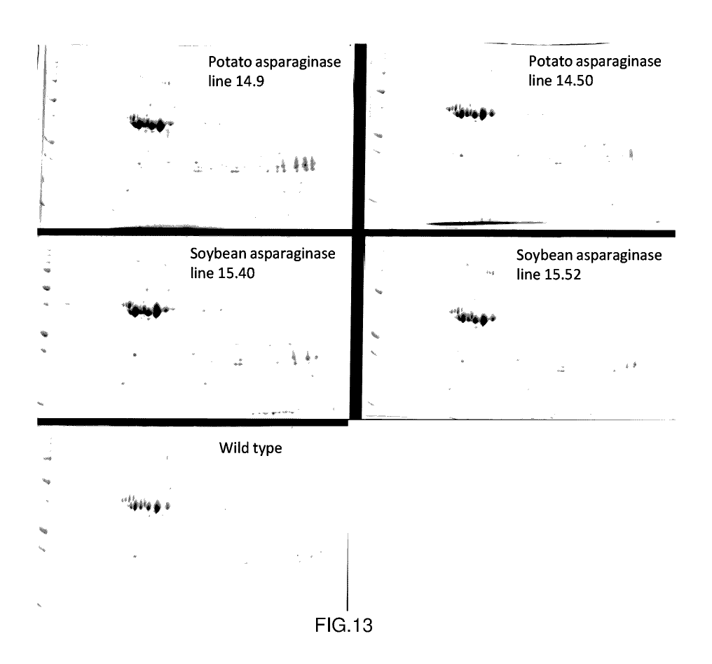

The transgenic tubers were further evaluated for free Asn content and scored in comparison with conventional cv. Atlantic tubers from plants grown side-by-side in the greenhouse. The tubers that exhibited high scores for either potato or soybean Asnase gene expression by RT PCR also exhibited a phenotype of reduced free Asn content (FIG. 12) Tubers from each generation were used to propagate the next generation of plants and tubers, and the study was repeated for multiple clonal generations with similar results.

The comparative yield of potato tubers derived from individual pots of the transgenics plants with non-transgenic Atlantic cv. was similar. Although all transgenic plant and the conventional cv. Atlantic plants yielded tubers of varying sizes, the overt morphology of the tubers appeared identical with no developmental or morphological differences as the result of the reduced Asn phenotype (FIG. 11). The interior appearance of conventional tubers and tubers of all of the transgenic lines exhibited the same color and other features, demonstrating that the conventional and transgenic tubers were identical in morphology, including plants that were grown though successive generations either as single plants grown in pots or in contained beds. No overt differences in the potato's growth, development, flowering time, or production of tubers were observed under any of the growth conditions.

Reduced Asn Content does not Alter the Relative Distribution of Potato Polypeptides.

The impact of reduced Asn content on potato tubers was examined by assaying total protein and starch assays, and showed little or no differences between transgenic and non-transgenic plant lines. Even the line that exhibited the greatest reduction free Asn, i.e., line 14-9 (FIG. 12) was determined to be essentially identical in starch and protein content to the conventional cv. Atlantic control. To further analyze the potential effects of reduced free Asn on the potato tuber's proteome, two-dimensional IEF/SDS-PAGE fractionation of proteins was performed (FIG. 13). The polypeptide profile of all four ectopic Asnase expressing lines were very similar to each other and to the conventional cv. Atlantic tuber's profile, indicating that the decrease in free Asn content and its role as NH₄ donor does not exert a significant effect on the diversity of proteins produced.

Non-Targeted Metabolomics Shows that Ectopic Asnase Expression Shifts Some Carbon Allocation to Glycans.

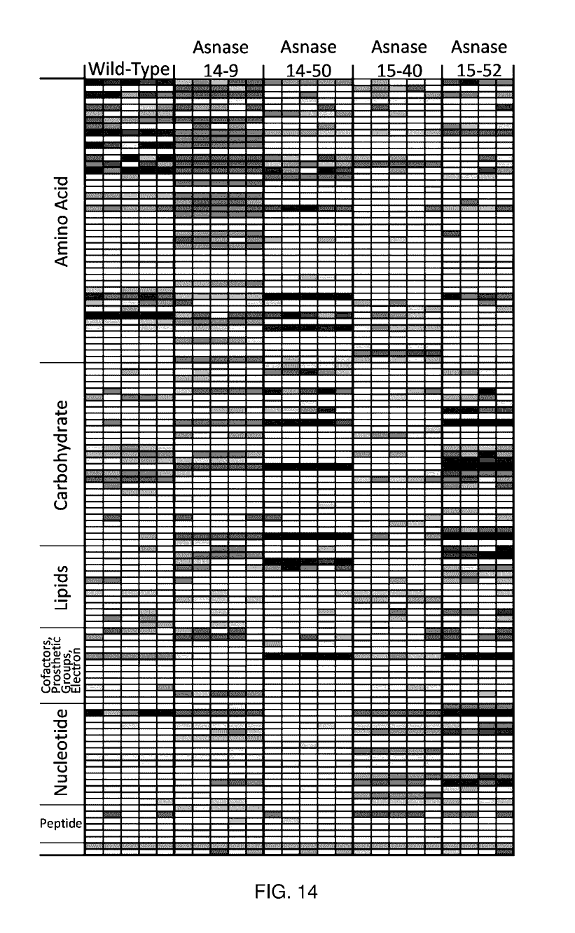

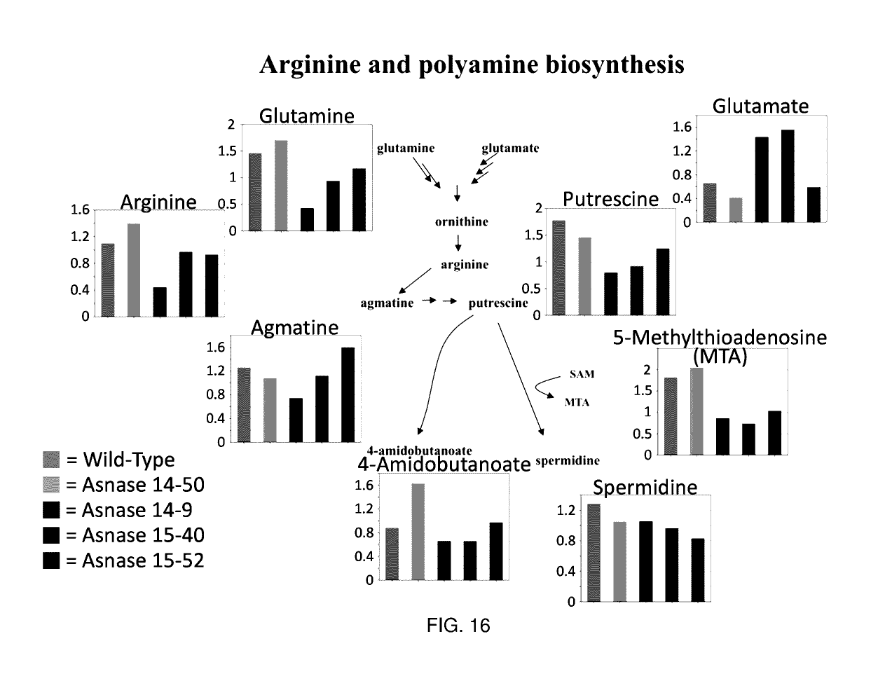

Asparagine is a major transporter of nitrogen in plants and a nitrogen donor into the amino acid pathways and related metabolites. A reduction in Asn content was therefore predicted to impact the abundance of other metabolites that are directly or secondarily derived from Asn nitrogen donor activity. To examine the abundance of metabolites in non-transgenic and transgenic potato tubers, non-targeted metabolomics analysis was performed. The results of this analysis are shown in FIGS. 14-16. FIG. 14 presents a heat map graphical representation of the total dataset. The tubers of each of the four transgenic ectopic Asnase-expressing lines exhibit a pattern of metabolite abundance different from that of tubers from (non-transgenic) conventional cv. Atlantic.

The compiled results of these studies indicated two overall patterns of metabolite content that result from ectopic expression of Asnase. The first of these patterns is illustrated by results of potato Asnase line 14-9 and the soybean Asnase line 15-40. The second pattern is illustrated by the similar results of potato Asnase line 14-50 and of soybean Asnase in line 15-52. Both of these patterns differ from the heat map metabolite profile of conventional Atlantic tubers. The similarity of the potato and soybean Asnase metabolite profiles indicated that the profile pattern is a consequence of the intrinsic activity of the overexpression of Asnase rather than a property of the plant source of the Asnase. The heat map of the 14-9 and 15-40 lines indicated a stronger phenotype than the 14-50 and 15-52 lines. For all of the ecotopic Asnase lines, the general features of the changes was a decrease in nearly all of the amino acids directly related to NH₄ donation, as illustrated in FIG. 5. The only exception was glutamate, which showed a significant increase in the amount of free amino acid.

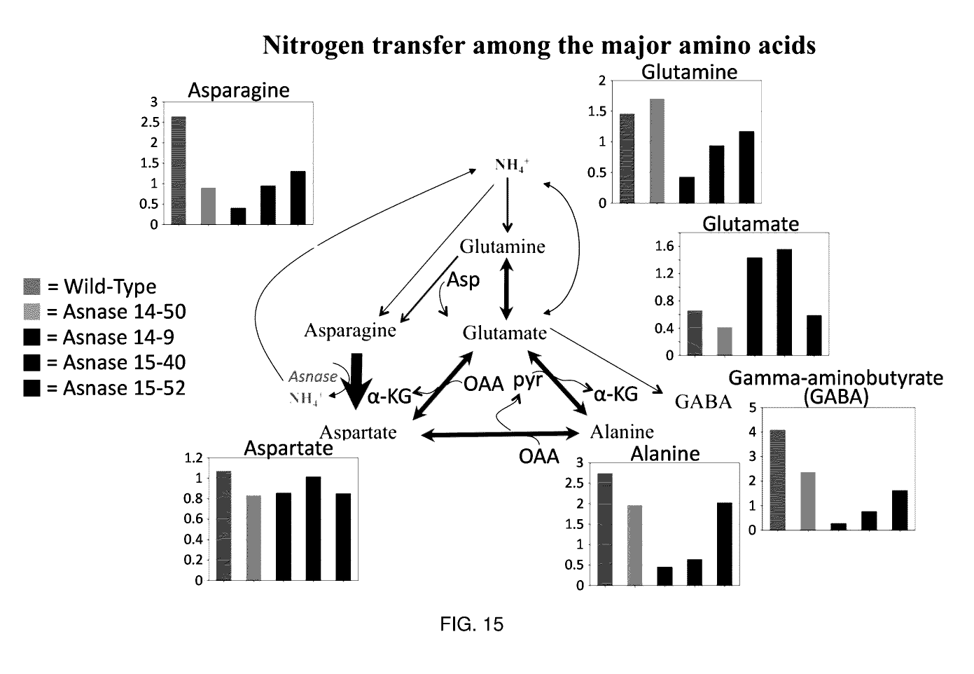

The ectopic expression of Asnase alters glutamine/glutamate content which in turn induced changes in arginine-related molecules, further amplifying the cascade effect on the metabolome as a consequence of the reduced nitrogen status of the transgenic tubers shown in FIG. 15.

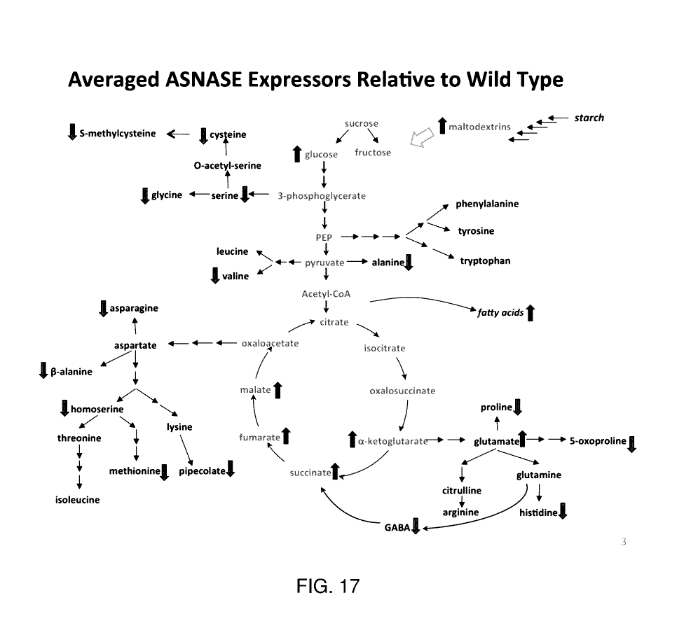

As a result of the decrease in content of amino acids and amino acid-related molecules, there was an overall shift in the abundance of non-nitrogenous molecules in intermediate metabolism that resulted in an increase in metabolites of the citric acid cycle and, further downstream, as an increase in the abundance of selected glycans. The interrelationship of alteration in carbon allocation is summarized in FIG. 17.

As shown in Example 2, systemic effects were observed in tobacco with constitutively expressed Asnase. In contrast, by targeting ectopic Asnase expression to a target organ—in this instance the tuber—Asn reduction was restricted to the target organ, and the overt plant and its agronomic properties were unaffected. Non-targeted metabolomics assessment of the potato tubers (FIG. 17) indicated a parallel physiology to the observations in systemic Asn reduction in the tobacco plants by showing an overall reduction in free amino acids and an overall increase in some free sugars.

Background non-targeted metabolomic assays on potatoes expressing ectopic Asnase indicated that the metabolic changes that occurred with reduced free Asn levels resulted in an increase in free fatty acid content, metabolically derived from sugars. This showed that, in addition to leveraging the Asn reduction trait into producing enhanced growth and value-added sugar/glycan derived products, the Asn reduction is also useful for reprogramming seeds for increased fatty acid and its derived oil.

Carbohydrate Analysis.

The carbohydrate composition of non-transgenic cv. Atlantic and transgenic tubers was profiled by services of the USDA Complex Carbohydrate Research Center (Athens, Ga.). The vast majority of the total carbohydrate of all of lines was in the form of starch and glucose. The transgenic lines exhibited slightly elevated levels of other glycans, including arabinose, galactose, mannose, and xylose compared with non-transgenic tubers, representing a small shift of carbon allocation to glycans other than starch. The increase in the glucose polymer malto-dextrins in the transgenic lines represents the re-allocation of carbon flux, although the total amount of maltodextrins is small compared to starch. The total glycan analysis and its glucose determinations did not show any significant differences in glucose abundance in excess of the variations between tubers.

An Increase in Asparaginase Activity in Potato Demonstrated Tuber-Specific Decrease in Asparagine and Decreased Propensity to Produce Acrylamide Upon Cooking the Tuber Under High Heat Conditions.

Free Asn is an important molecule for the food processing industry, as Asn and sucrose under the high heat of industry processing conditions produces acrylamide in processed foods. Acrylamide is a potential carcinogen and a substance of continuing interest to regulators. Asnase, as a fungal-derived industrial enzyme, is currently used to treat potato and wheat flour prior to its fabrication into processed foods. Potatoes produced as in this example can produce reduced acrylamide in response to high heat.

Seed-Specific Ectopic Asnase Expression Shifts Seed Metabolism.

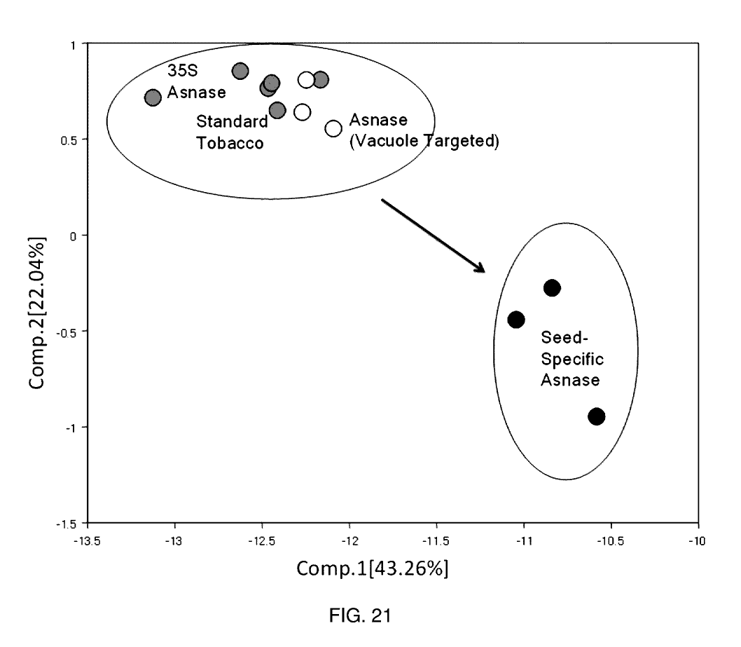

Principle component analysis mapping showed a shift in metabolomics profiles in tobacco seeds comprising ectopic Asnase under the control of a seed-specific promoter. Neither a vacuole-specific promoter or the ubiquitous 35S promoter showed any shift in metabolism in tobacco seeds relative to wild-type tobacco seeds (FIG. 21).

### Example 4

### Elevated Sugar and Solids Accumulation in Tomatoes

As described herein, carbon reallocation is used to create an elevated sugar and solids accumulation trait in processing tomatoes, thereby increasing their economic performance. The materials and methods described herein accelerated the plant's life-cycle and growth rate to increase their economic performance, especially under facility-intensive growth in hydroponics used for fresh tomatoes. For tomato crops, the 35S promoter was utilized to accelerate growth and life cycle and the E8 fruit-specific promoter was utilized to produce sweeter fruit having increased carbonaceous solids.

Ectopic Asnase expression was used to increase free sugar levels. As in potato crops, the Asnase expression induced a significant increase in free sugar and altered overall carbon allocation. Higher sugar content is a desirable trait in tomato crop products. The shift in carbon allocation to sugars and their polymers was primarily a response to the reduction of free Asn resulting from ectopic Asnase expression.

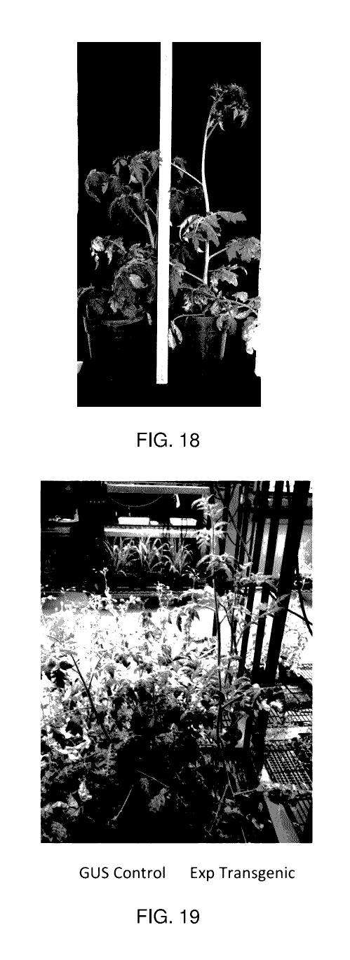

T0 seeds of tomato plants expression ectopic Asnase were produced using the same 35S construct used in tobacco in Example 2. These systemic Asnase-expressing tomatoes were subject to recurrent selection to obtain homozygous plants for trait evaluation, including both overt plant growth and fruit composition. Resulting tomato plants exhibiting increases in overall growth (FIGS. 18-19), elaborated root growth, and increased carbon fixation activity were selected for positive yield. Paralleling the results seen in tobacco, ectopic Asnase expression in tomatoes increased free sugars and carbonaceous solids, including in the fruit.

Paralleling the tuber-specific potato results, the overt trait in tomato was enhanced by Asnase-induced carbon rebalancing. To specifically alter fruit composition, new constructs were transferred to tomato, where the Asnase gene was controlled by the E8 tomato ripening-specific, fruit-specific promoter (see, e.g., Hirai et al. (2011), Transgenic Research, 20: 1285). As with the 35S-regulated construct, these transgenes were transferred to tomato, and plants were selected and regenerated and subjected to recurrent selection to obtain homozygous lines. The tomatoes produced by the 35S promoter or the E8 fruit-ripening-specific promoter, along with parallel controls, were assessed for overt plant and fruit development and composition and, more specifically, for changes in free sugar and carbonaceous solid content and composition. Tomato lines were analyzed in detail using selected—omics assessment, emphasizing metabolomic changes in comparison to controls and to the extant tomato metabolomic literature (Allwood et al. (2011), Methods Enzymol., 500:299; Bono et al. (2004); Tieman et al. (2012), Current Biology, 22: R443). Homozygous tomato lines characterized for enhanced sugar/solids trait were then used for expansion and evaluation in agronomic contexts.

Tomato plants transformed with an Asnase expression construct showed accelerated growth when compared to sham-transformed control (GUS) (FIG. 19). The photosynthetic carbon rate was increased by at least ˜25%.

### Example 5

### Increasing Free Sugars and Growth Rate in Carrots

Translating the increased free sugars and growth rate increases from Asnase expression enhances taproot value. Taproots and tuberous roots are a major source of carbonaceous products with important examples of taproots including carrots and sugar beets, and tuberous roots include some of the global great crops including cassava, yam, and sweet potato. The output traits for all of these crops are carbonaceous, primarily starch and cell wall mass. For some the free sugars are an important component, or the value-trait, as for sugar beet. The increases in carbonaceous output traits resulting from ectopic Asnase, whether systemic with increases in carbon fixation, plant growth rate, and increased flux into cellulose/free sugars, or as an organ-specific with increases in sugars and starch without altering tuber morphology, enhances these crops.

Carrots are transformed using Agrobacterium transformation of somatic embryos with a taproot specific promoter, e.g. the Sweet potato SRD1 (Noh et al. (2012), Transgenic Res., 21:265; Hardegger and Strum (1998), Mol. Breeding, 4: 119). For carrot crops, a taproot-specific promoter is used for the desired trait of a sweeter tap root, and the 35S promoter is utilized for increased growth rate in tap root.

### Example 6

### Enhanced Starch, Oil, and Lifecycle Traits

Reprogramming carbon allocation into starch and oil carbonaceous sinks and accelerating growth rates would be advantageous for a double winter crop. The Brassicas, of which Camelina is a member, are major oil and protein sources and a competitive product with soybean. Soybean storage products are dominantly localized in the cotyledons while Brassicas have a prominent endosperm and embryo, each substantial contributors to the overall storage substance profile. Seed endosperm is preferentially specialized to produce carbonaceous sink (starch, galactomannans, oil) storage substances over nitrogenous protein sinks. Camelina is a commercial, relatively fast oil seed capable of being grown in more marginal environments than, for instance, soybean. Camelina research uses the vast Brassica (Arabidopsis) toolkits and databases including facile flower transformation and the available genome database. In some regions, including much of the US Southwest, it would be feasible to double crop Camelina during the winter months if the plant's life cycle could be accelerated by 1 week (two weeks total for two crops).

Asnase, as a constitutive expressed transgene, accelerates lifecycle enabling double-crop potential. In addition, the shift toward carbonaceous substances, whether starch/cellulose or seed oil, are value-enhancing traits. To test the potential of accelerating the life cycle and/or enhancing carbonaceous substance output, both constitutive (35S) and seed-specific (oleosin promoter) promoter-regulated Asnase are transferable to Camelina. Camelina transformation is demonstrated in, for example, Herman and Schmidt (2010), using gene expression cassettes with promoters for Camelina. In Camelina, the 35S promoter is utilized for accelerated life cycle, and Camelina oleosin seed-specific promoter may be utilized for increased carbonaceous sinks specifically in seeds.

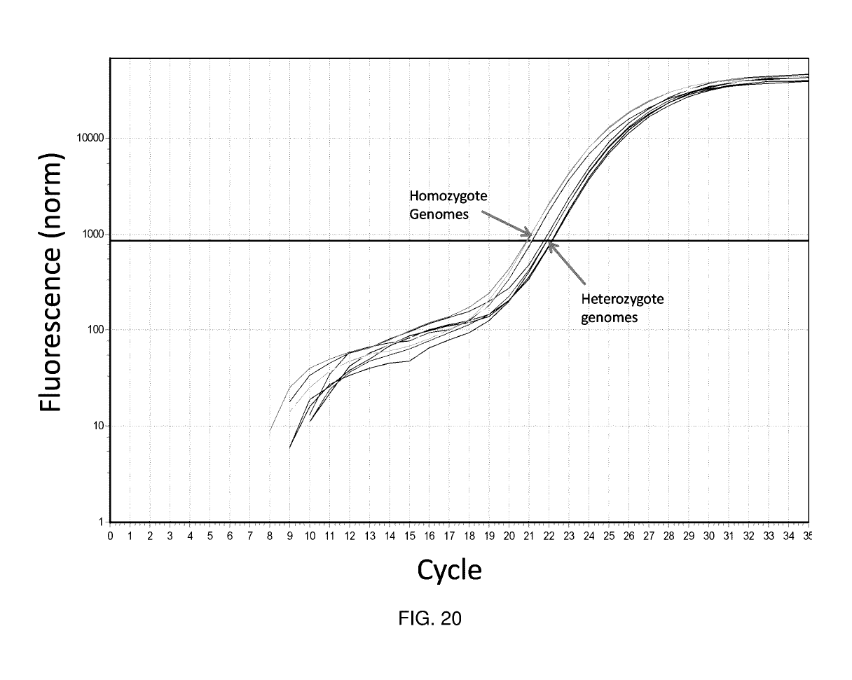

Soybean may be transformed according to methods known in the art (e.g., U.S. Pat. No. 5,164,310). To test the potential of accelerating the life cycle and/or enhancing carbonaceous substance output, both constitutive (35S) and seed-specific (glycinin promoter) promoter-regulated Asnase are transferable to soybean. Ectopic Asnase has been successfully introduced and expressed in soybean in a tissue-specific manner utilizing the nucleic acid sequences described herein and the glycinin seed-specific promoter (FIG. 20).

### Example 7

### Increasing Content of Fermentable Carbon in Maize and Switchgrass

Increasing the content of fermentable carbon in maize provides an example of a monocotyledonous plant for enhancing biomass end use. Maize is one of the global great crops and a large fraction of US maize production is destined for bioethanol production. Maize stover (the stalk, leaf, husk, and cob remaining after the harvest of the grain) cell walls are a significant biomass contributor and may be further enhanced by use of the materials and methods described herein. Even a small or marginal increase in glycan polymers per unit mass in stover can be leveraged to greatly increase output product (ethanol). The advantageous carbonaceous phenotype of increased cellulose and increased carbon fixation in a monocotyledonous crop already used for biomass has good commercial value. Higher levels of cellulosic content add considerable efficiency and value to stover biomass conversion to ethanol.

Maize 35S promoter is utilized for increasing overall plant carbonaceous sinks. Transgenic maize containing the 35S promoter regulated Asnase have been created in H99 using Agrobacterium transformation and is introgressed in B73. This step requires several generations (about 120 days each) to complete.

Using the Asnase trait in other biomass grasses, such as Brachypodium and Switchgrass, has further beneficial traits. Similarly, the materials methods herein have application to fodder plants.

### Example 8

### Increasing Oil and/or Fermentable Carbohydrates in Algae

Algal cells in culture can be induced to accumulate excess oil by reducing extrinsic (media) nitrogen availability, thereby decreasing the algal cell's nitrogen status and causing a shift toward oil accumulation. Algae with high Asnase expression were observed to have a higher photosynthetic rate (i.e. absorb more carbon) and accumulate greater oil content. The oil can be harvested for biofuel production, while the increased metabolic efficiency can be harnessed as a bioreactor to scrub CO₂ from the atmosphere/effluents. This is particularly advantageous since algae can grow in excess CO₂ environments.

Algal cells are transformed with the nucleic acids described herein by methods well known in the art, the expression of which can be driven by promoters also known in the art. For examples of methods and promoters useful for transforming algal cells, please see Hallmann (2007), Transgenic Plant Journal, 1(1):81.

As described herein, transgenic plants expressing ectopic soybean or potato cytoplasmic Asnase accumulate proportionally higher cellulose, starch, and free-sugars compared to controls by reconfiguring source and sink composition to favor carbonaceous molecules and compounds. Reducing cytoplasmic Asn induces increased carbon fixation and its assimilation to sugars and glycan-polymers. Through this strategy, it has been shown that plants expressing ectopic Asnase enhance the rate of carbon fixation, resulting in the increase of carbonaceous substances in the plants. This engineering strategy, that reduction of free Asn produces enhanced carbonaceous traits, has applications in crops with fruit, taproot, biomass, and oil output traits and products. The examples described provide models for the engineering strategy in the context of individual specific output traits relevant to these and to other major crops.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiments disclosed herein contemplated for carrying out this invention, but that the invention will include all embodiments falling within the scope of the claims.

1. A method for redirecting a plant's allocation of fixed carbon toward carbohydrate polymers relative to a control plant, comprising increasing expression in a plant at least a first copy of a nucleic acid having at least 70% sequence identity to the amino acid sequence of SEQ ID NO: 2 sufficient to redirect the plant's allocation of fixed carbon toward carbohydrate polymers relative to a control plant.

2. (canceled)

3. (canceled)

4. (canceled)

5. The method of claim 1, wherein the fixed carbohydrate comprises in at least one form of carbohydrate polymer selected from the group consisting of: sugars; starch; cellulose; and oil.

6. The method of claim 1, wherein the redirection of the plant's allocation of fixed carbon towards carbohydrate polymers relative to a control plant occurs under non-stress conditions.

7. (canceled)

8. (canceled)

9. (canceled)

10. (canceled)

11. (canceled)

12. (canceled)

13. The method of claim 1, wherein the first copy of the nucleic acid is operatively linked to a first promoter, and further comprising introducing and expressing a second copy of the nucleic acid in the plant, wherein the second copy of the nucleic acid is operably linked to a second promoter.

14. The method of claim 13, wherein one copy of the nucleic acid is operably linked to a tissue non-specific promoter and the other copy of the nucleic acid is operably linked to a tissue-specific promoter.

15. (canceled)

16. The method of claim 1, wherein the plant is selected from the group consisting of: soybean; potato; tomato; tobacco; Camelina spp.; maize; carrot; switchgrass; sugar beet; cassava; sweet potato; yam; Brachypodium; onion; safflower; sunflower; canola (rapeseed); hemp; cotton; sesame; peanut; flax; rice; wheat; oats; and algae.

17. A plant or plant part obtained by the method of claim 1.

18. (canceled)

19. (canceled)

20. (canceled)

21. (canceled)

22. (canceled)

23. A construct comprising a first copy of a nucleic acid encoding a polypeptide having at least 70% sequence identity to the amino acid sequence of SEQ ID NO: 2, one or more control sequences capable of driving expression of the first copy of the nucleic acid, and optionally a transcription termination sequence.

24. The construct of claim 23, further comprising a second copy of the nucleic acid sequence and one or more control sequences capable of driving expression of the second copy of the nucleic acid, wherein the one or more control sequences capable of driving expression of the second copy of the nucleic acid are different from the one or more control sequences capable of driving expression of the first copy of the nucleic acid sequence.

25. (canceled)

26. (canceled)

27. (canceled)

28. A plant, plant part, or plant cell comprising the construct of claim 23.

29. The plant of claim 28, or a plant cell or plant part derived thereof, wherein said plant is selected from the group consisting of: soybean; potato; tomato; tobacco; Camelina spp.; maize; carrot; switchgrass; sugar beet; cassava; sweet potato; yam; Brachypodium; onion; safflower; sunflower; canola (rapeseed); hemp; cotton; sesame; peanut; flax; rice; wheat; oats; and algae.

30. Harvestable parts of the plant of claim 28, wherein the harvestable parts are selected from the group consisting of: shoot biomass; fruits; roots; taproot;
and seeds, and wherein the harvestable parts comprise the construct.

31. A method for making a plant having altered fixed carbon allocation relative to a control plant, comprising transforming a plant, plant part, or plant cell with the construct of claim 23.

32. (canceled)

33. (canceled)

34. (canceled)

35. (canceled)

36. (canceled)

37. (canceled)

38. The transgenic plant of claim 31, or a transgenic plant cell or transgenic plant part derived thereof, wherein said plant is selected from the group consisting of: soybean; potato; tomato; tobacco; Camelina spp.; maize; carrot; switchgrass; sugar beet; cassava; sweet potato; yam; Brachypodium; onion; safflower; sunflower; canola (rapeseed); hemp; cotton; sesame; peanut; flax; rice; wheat; oats; and algae.

39. (canceled)

40. (canceled)

41. (canceled)

42. (canceled)

43. (canceled)

44. (canceled)

45. (canceled)

46. (canceled)

47. A method for scavenging environmental CO₂, comprising increasing expression in algae a nucleic acid encoding a poly nucleotide having at least 70% sequence identity to the amino acid sequence of SEQ ID NO: 2, and scavenging environmental CO₂.

48. (canceled)


SEQUENCE LISTING (2 sequences)

SEQ ID NO: 1 - DNA, 980 bp, Glycine max
atgggagggtgggctatcgcggtgcatggtggcgctggtgtcgaccctaatctcccactcgagcaccaagaagaagcgaaacaacttctcacgcgctgtctcaatcttggaatctctgctctaaattctaatcttcccgccatagatgtcgtcgaacttgtggtgagggaattggaaacggatccgctgttcaattcgggccgtggatctgccctgacagaaaaaggaacggtggagatggaggctagcatcatggatggtccaaaacgacgatgcggcgccgtttcgggtcttaccaccgttaagaacccgatctcgcttgctcgtttggttatggacaaatccccgcattcctacctcgccttttccggcgccgaagaatttgctaggcaacagggtgtggaggttgtggacaacgagtactttatcacacctgacaacgttggcatgctcaagctggcaaaggaagcaaacacaatcctgtttgattaccgaatcccaacatcggggtacgaaacgtgcggatccggtgtggagagtccactgcaaatgaatggactcccaataagcgtgtacgcgcccgaacccgttgggggcgtggtggttgaccggaagggccggtgcgcggcagcaacatccacgggagggctgatgaacaaaatgaccgggcgcatcggtgattcgccgctgataggcgccgggacctacgcgtgcgacgtgtgcggcgtatcgtgcacgggtgaaggcgaggccatcatacgtggcacgctggcgcgtgaggtggcagcggtgatggaatacaaaggcttgaagcttcaagaagccgtggattttgtgatcaagcaccgtctcgatgaagggatggctggtctcattgctgtgtccaacgctggggaagtggcttatggtttcaattgcaatggcatgtttaggggctgtgccactgaaaatggcttcatggaggtcggaatctgggaata

SEQ ID NO: 2 - protein, 326 aa, Glycine max
MGGWAIAVHGGAGVDPNLPLEHQEEAKQLLTRCLNLGISALNSNLPAIDVVELVVRELETDPLFNSGRGSALTEKGTVEMEASIMDGPKRRCGAVSGLTTVKNPISLARLVMDKSPHSYLAFSGAEEFARQQGVEVVDNEYFITPDNVGMLKLAKEANTILFDYRIPTSGYETCGSGVESPLQMNGLPISVYAPEPVGGVVVDRKGRCAAATSTGGLMNKMTGRIGDSPLIGAGTYACDVCGVSCTGEGEAIIRGTLAREVAAVMEYKGLKLQEAVDFVIKHRLDEGMAGLIAVSNAGEVAYGFNCNGMFRGCATENGFMEVGIWE